(19) United States
(12) Patent Application Publication
(10) Pub. No.: US 2024/0345075 A1
(43) Pub. Date: Oct. 17, 2024

(54) METHODS OF DIAGNOSING, PROGNOSING, AND TREATING MULTISYSTEM INFLAMMATORY SYNDROME IN CHILDREN (MIS-C) AND SEVERE COVID-19, AND APPARATUSES THEREOF

(71) Applicant: Rutgers, The State University of New Jersey, New Brunswick, NJ (US)

(72) Inventors: Maria L. Gennaro, New Brunswick, NJ (US); Jeisac Guzmán Rivera, New Brunswick, NJ (US); Dirk Foster Moore, New Brunswick, NJ (US)

(21) Appl. No.: 18/606,510

(22) Filed: Mar. 15, 2024

Related U.S. Application Data
(60) Provisional application No. 63/604,578, filed on Nov. 30, 2023.
(60) Provisional application No. 63/452,850, filed on Mar. 17, 2023.

(51) Int. Cl. G01N 33/543; G01N 33/577; G01N 33/68

(52) U.S. Cl. CPC G01N 33/54366; G01N 33/577; G01N 33/6893; G01N 2800/24

(57) ABSTRACT

Embodiments disclosed here are directed to novel methods for diagnosing MIS-C or severe acute COVID-19, by detecting a biomarker in a blood sample from a subject suspected of or suffering from MIS-C or severe acute COVID-19, where the methods are for early detection or diagnosis of MIS-C or severe acute COVID-19. Also, disclosed are embodiments directed to apparatuses for use with the novel methods described here. Methods of detecting at least one biomarker and/or diagnosing MIS-C or severe acute COVID-19 disease in a pediatric subject suspected from suffering MIS-C or severe acute COVID-19 that can be used in combination with methods of treating a subject suspected of or suffering from MIS-C or severe acute COVID-19, accelerates the diagnosis and treatment, and improves prognosis.

### CROSS-REFERENCE TO RELATED APPLICATIONS

This application claims the benefit of and priority to U.S. Provisional Application No. 63/604,578, filed Nov. 30, 2023, and also claims the benefit of and priority to U.S. Provisional Application No. 63/452,850, filed Mar. 17, 2023, which are incorporated herein by reference in its entirety.

### STATEMENT REGARDING FEDERALLY SPONSORED RESEARCH OR DEVELOPMENT

This invention was made with government support under Grant No. R61 HD105619 awarded by the National Institutes of Health. The government has certain rights in the invention.

### FIELD OF DISCLOSURE

The present disclosure in various embodiments described here is directed to methods and apparatuses for diagnosing, prognosing, and treating Multisystem Inflammatory Syndrome in Children (MIS-C) or severe acute COVID-19 caused by a SARS-COV-2 infection in a subject.

### BACKGROUND

Severe clinical manifestations of SARS-COV-2 infection are thought to be less frequent in children than in adults, for multiple reasons, including availability of the ACE-2 receptor, cross reactive antibodies primed by prior coronavirus infections, and a heightened innate immune response. However, severe manifestations with fatal consequences can still occur in children. As for adult infection, the severity of pediatric COVID-19 has been ascribed to genetic predisposition, comorbidities, and sociodemographics. Severe COVID-19 typically manifests as respiratory distress, cardiac dysfunction, coagulopathy, and widespread inflammation; its treatment includes oxygen supplementation, steroids, and immunomodulators. In a small number of cases, however, pediatric SARS-COV-2 infection evolves into a post-acute syndrome called Multisystem Inflammatory Syndrome in Children (MIS-C), which is characterized by the involvement of at least two organ systems, including gastrointestinal, cardiovascular, and neurological, and that, if left untreated, may result in multiorgan failure. MIS-C typically manifests 3-6 weeks after infection and can occur even after asymptomatic or mild acute infection. MIS-C symptoms resemble those observed in other pediatric conditions, including Kawasaki disease, sepsis and Toxic Shock Syndrome. However, gastrointestinal and cardiovascular manifestations tend to be more prominent in MIS-C relative to these other conditions. While the underlying causes of MIS-C are still poorly defined, genetic predisposition, presence of autoantibodies, and presence of viral antigen in blood may play a causative role. Treatment normally involves administration of intravenous immunoglobulins, steroids, and immunomodulators. Since MIS-C is diagnosed by a process of elimination of other conditions or diseases, there is a need for early diagnosis and proper treatment of MIS-C. The sensitivity of available methods to diagnose MIS-C or severe acute COVID-19 is limited, which delays the critical timing for effective therapy. Accordingly, there is a pressing need for a more sensitive assay for early detection and diagnosis of MIS-C or severe COVID-19 in a subject in, for example, the pediatric population.

### SUMMARY

In accordance with the objectives disclosed herein, embodiments of the present disclosure satisfy the aforementioned needs and provide related advantages as well. The present disclosure provides in some objects, methods and apparatuses defined by embodiments of the disclosure and in connection with examples provided here. Other features and advantages of various embodiments of the disclosure will be apparent from the description.

As described here, the present disclosure features methods or uses comprising detecting at least one biomarker (e.g., Fc gamma binding protein (FCGBP); Mannose receptor C-type 1 (MRC1); LPS binding protein (LBP); zonulin) in a sample from a subject suspected of or suffering from Multisystem Inflammatory Syndrome in Children (MIS-C) and/or severe acute COVID-19, methods of diagnosing and/or prognosing MIS-C and/or severe acute COVID-19 in a sample from the subject, methods of treating a subject suffering from MIS-C and/or severe acute COVID-19, and apparatuses comprising a plurality of antibodies for detecting at least one biomarker associated with MIS-C and/or severe acute COVID-19 for use in the methods disclosed herein.

The inventive disclosure is based, at least in part, on the discovery of biomarkers that are indicative of a diagnosis of MIS-C, or severe acute COVID-19 in a subject (e.g., a human, an infant, a child, a teenager, an adult of less than 21 years). Accordingly, antigens and/or ligands in a subject are present in a subject suffering from MIS-C, or severe acute COVID-19. Moreover, blood samples (e.g., serum, plasma) from a subject suffering from MIS-C, or severe acute COVID-19 contain biomarkers (e.g., wherein at least one biomarker comprises a function selected from the group consisting of: mucosal immunity (see, e.g., R. Bansil and B.S. Turner. Adv Drug Deliv Rev. 2018 Jan. 15; 124:3-15; P. Brandtzaeg. Scand J Immunol. 2009 December; 70(6):505-15.; J.M. Creeth. Br Med Bull. 1978 January; 34(1):17-24) (e.g., mucins, Fc gamma binding protein (FCGBP), zonulin, immunoglobulins (e.g., IgA. IgM), antimicrobial proteins (e.g., defensins, lysozyme), transferrins (e.g., lactoferrin)); macrophage activation (Moin et al. Sci Rep. 2021 Mar. 19; 11(1):6428; Shaked et al. Arterioscler Thromb Vasc Biol. 2014 May; 34(5):1085-92) (e.g., Mannose receptor C-type 1 (MRC1), CD163, CD14, Gal-3BP, matrix metalloproteinases (e.g., MMP7, MMP9), cytokines); immune response enhancement (e.g., white blood cell count, erythrocyte sedimentation rate, procalcitonin (sec, e.g., Vijayan et al. J Intensive Care. 2017 Aug. 3; 5:51), C-reactive protein (CRP), serum amyloid A (SAA), cytokines, lipopolysaccharide (LPS) binding protein (LBP), Fc gamma binding protein (FCGBP), Mannose receptor C-type 1 (MRC1)); fatty acid binding protein (FABP), glucagon-like peptide (GLP)-2, citrulline, lipopolysaccharide (LPS), LPS-binding protein (LBP); IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; and any combinations thereof). Methods of the disclosure also include sensitive ELISA methods to detect at least one biomarker described here in a blood sample for early diagnosis of MIS-C, or severe acute COVID-19.

### BRIEF DESCRIPTION OF FIGURES

FIG. 1A shows a plot of the first two principal components with the two groups (MIS-C and mild/asymptomatic SARS-COV-2 infection) coded as red and blue, respectively, and the biomarkers using gene names. Principal component analysis is a mathematical algorithm for reducing the dimensionality of a data set while maintaining most of the information it contains. In this case, the first two principal components explain 36.1 percent and 24.4 percent, respectively, in the data variability for the 23 proteins listed in FIG. 1B. In this figure, the red points represent subjects in the MIS-C group, and the blue dots subjects in the mild/asymptomatic group. FIG. 1B provides a list of the proteins (Description) identified as gene (protein) names in FIG. 1A, their estimated log2 differences (coeff), and p-values (pVal).

FIG. 2 illustrates a Receiver Operating Characteristic (ROC) curve for the true positive rate of the predicted classifications of MIS-C and severe acute SARS-COV-2 infection versus the false positive rate. The support vector machine model based on the 23 proteins described here has an Area Under the Curve (AUC)=0.9689.

FIGS. 3A-3B provides exemplary schematics of ELISA protocols used in various embodiments described here. FIG. 3A shows a direct sandwich ELISA: (1) immobilized capturing antibody against any of the biomarkers; (2) addition of antigen (solid circle) from subject blood sample; (3) conjugated detection antibody (antibody conjugated with detectable label (star); (4) substrate (droplet) addition, signal detection, and quantification, where washes are performed to remove excess antigen after (2) or excess conjugated detection antibodies after (3). FIG. 3B presents a competitive ELISA: (1) immobilized capturing antibody against any of the biomarkers; (2) addition of antigen (solid circle) and antigen conjugate (solid circle with detectable label (star)), which competitively bind to form an antigen enzyme complex and/or a detectable antigen-conjugated enzyme complex from subject blood sample, where washes are performed to remove excess antigen or antigen conjugates after (2); (3) substrate (droplet) addition, signal detection, and quantification. FIG. 3C shows that signal strength (Y-axis) is inversely proportional to the concentration of the analyte (X-axis) in competitive ELISA or other competitive immunoassays.

FIGS. 4A-4B present an exemplary summary of a work plan of a cohort study described here. FIG. 4A shows a flowchart for a cohort study and nested case-control study, where children and adolescents presenting with suspected COVID-19 or MIS-C at an Emergency Department (ED) are recruited, consented, and plasma collected at the time of initial blood draw. FIG. 4B illustrates a summary of biomarker measurements for cases and controls performed in a blinded fashion.

FIG. 5A provides a flowchart incorporating the subject testing of biomarkers in determining MIS-C and/or or severe acute COVID-19 in a pediatric subject. FIG. 5B presents a treatment plan incorporating the subject testing of biomarkers in determining MIS-C and/or severe acute COVID-19 in a pediatric subject.

### DETAILED DESCRIPTION

Detailed embodiments of the present disclosure are described herein; however, it is to be understood that the disclosed embodiments are merely illustrative of the disclosure that may be embodied in various forms. In addition, each of the examples given in connection with the various embodiments of the disclosure is intended to be illustrative, and not restrictive.

Multisystem inflammatory syndrome in children (MIS-C) is an inflammatory condition, where the heart, lungs, kidneys, brain, skin, eyes, and/or gastrointestinal organs are inflamed. Although the causes of MIS-C are yet to be identified, many children, teens, or individuals younger than 21 years of age with MIS-C were infected with the SARS-COV-2 virus, or had been in close contact with someone infected with the SARS-COV-2 virus.

Disclosed herein are embodiments directed to novel methods for detecting at least one biomarker in a sample (e.g., blood, plasma, serum) from a subject suspected of or suffering from Multisystem Inflammatory Syndrome in Children (MIS-C) or severe acute COVID-19 for early and/or sensitive detection of MIS-C or severe acute COVID-19. Also, disclosed are embodiments directed to apparatuses for use with the novel methods described here and methods of treating MIS-C, or severe acute COVID-19 in a subject, where the subject is a child, teen, or human of less than 21 years of age.

### Method of Detecting and Treating MIS-C or Severe Acute COVID-19

In some embodiments, the disclosure is directed to a method of detecting the presence of at least one biomarker in a subject, where the subject is suspected of suffering from Multisystem Inflammatory Syndrome in Children (MIS-C) or severe acute COVID-19. For example, the disclosure comprises methods of early detection of MIS-C or severe acute COVID-19 in a subject by exploiting proteins that are present in those suffering from MIS-C or severe acute COVID-19. One embodiment is directed to a method, comprising: (a) contacting at least one capture antibody against at least one biomarker disclosed here and a sample (e.g., blood; plasma; serum) from a subject suspected of or suffering from MIS-C or severe acute COVID-19, thereby forming a contacted sample on an apparatus, where at least one capture antibody binds at least one biomarker of the sample; (b) binding the contacted sample and a detection entity that binds to at least one biomarker of the sample; (c) detecting the detection entity, wherein the detection entity reflects the presence of at least one biomarker for Multisystem Inflammatory Syndrome in Children (MIS-C) or severe acute COVID-19 in the subject; and (d) treating the subject for Multisystem Inflammatory Syndrome in Children (MIS-C) or severe acute COVID-19, wherein at least one biomarker is selected from the group consisting of: FCGBP, MRC1, LBP, CD163, zonulin, and/or at least one biomarker comprises a function selected from the group consisting of: mucosal immunity (e.g., mucosal immunity (e.g., mucins, Fc gamma binding protein (FCGBP), immunoglobulins (e.g., IgA, IgM), antimicrobial proteins (e.g., defensins, lysozyme), transferrins (e.g., lactoferrin)); macrophage activation (e.g., Mannose receptor C-type 1 (MRC1), CD163, CD14, Gal-3BP, matrix metalloproteinases (e.g., MMP7, MMP9), cytokines); bacterial infectivity (see, e.g., A. Mohan and J. Harikrishna. Indian J Med Res. 2015 March; 141(3):271-3) (e.g., white blood cell count, erythrocyte sedimentation rate, procalcitonin, C-reactive protein (CRP), serum amyloid A (SAA), cytokines, lipopolysaccharide (LPS) binding protein (LBP)); intestinal damage/gut leakage (e.g., zonulin, fatty acid binding protein (FABP), glucagon-like peptide (GLP)-2, citrulline, lipopolysaccharide (LPS), LPS-binding protein (LBP)) (see, e.g., I. Schoultz and A. Keita. Cells. 2020; 9(8):1909); any biomarker identified in TABLE 1: IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; and any combinations thereof. In additional embodiments, such methods as described here are directed to combinations of biomarkers, where if the biomarker of (5) is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Further embodiments provide for such methods as described here, where the at least one biomarker comprises Macrophage mannose receptor 1/Mannose receptor C-type 1 (MRC1) and/or Tryptophan-tRNA ligase, cytoplasmic (WARS1), cither alone or in combination with at least one biomarker (or at least one biomarker comprising a function) selected from the group consisting of a biomarker of: Group (A) of TABLE 3 and/or a biomarker of Group (B) of TABLE 4 or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Other embodiments are directed to such methods described here where the at least one biomarker is selected from any one of Group (B) of TABLE 4, cither alone or in combination with any one of Group (A) of TABLE 3. Some embodiments provide for biomarkers that are directed primarily to detection of MIS-C (see, e.g., TABLE 1, #1-19), while other biomarkers are directed to MIS-C and/or severe acute COVID-19 (see, e.g., TABLE 1, #20-23). In some aspects, the apparatus of the method described here comprises at least one capture antibody, where the capture antibody is against at least one biomarker described here. The apparatus comprises one or more capture antibodies against a panel of biomarkers described here. Additional aspects are directed to such methods, where the at least one capture antibody can be configured to detect the at least one biomarker for diagnosing and/or prognosing MIS-C or severe acute COVID-19. In further aspects, the methods disclosed here utilize a subject sample, where the sample comprises at least one biomarker for Multisystem Inflammatory Syndrome in Children (MIS-C) or severe acute COVID-19 in the subject. Additional aspects of the described methods further comprise reducing background interference (e.g., blocking; washing) on the apparatus. Reducing background interference can occur by washing the apparatus comprising the contacted sample after (a) contacting at least one capture antibody and the sample (e.g., blood; plasma; serum) from the subject suspected of or suffering from Multisystem Inflammatory Syndrome in Children (MIS-C) or severe acute COVID-19; after (b) binding the contacted sample and the detection entity that binds to at least one biomarker; or both before and after (b) binding the contacted sample and the detection entity that binds to at least one biomarker. Other aspects of the method described here provide the detection entity comprising specificity for the at least one biomarker or specificity for the at least one capture antibody. The detection entity comprises a detectable label, where the detectable label is selected from the group consisting of: an enzyme label, a fluorescent label, and a biotin label. In some aspects, methods of the disclosure are provided, where the detectable label is selected from the group consisting of: horseradish peroxidase (HRP), alkaline phosphatase (AP). β-galactosidase, glucose oxidase, glucose-6-phosphate dehydrogenase, β-N-acetylglucosaminidase, invertase, xanthine oxidase, biotin, Fluorescein Isothiocyanate (FITC), Tetramethylrhodamine-5-(and 6)-isothiocyanate (TRITC), Alexa Fluor® (AF) dye (e.g., AF 350, AF 405, AF 430, AF 488, AF 500, AF 514, AF 532, AF 546, AF 555, AF 568, AF 594, AF 610, AF 633, AF 635, AF 647, AF 660, AF 680, AF 700, AF 750, AF 790), Allophycocyanin (APC), phycoerythrin (PE), Brilliant violet (BV) dye (e.g., BV 421, BV 480, BV 510, BV 570, BV 605, BV 650, BV 711, BV 750, BV 785), Peridinin-chlorophyll-protein (PerCP), Pacific blue, Pacific orange, PE/Dazzle 594, APC Fire 750, Super Bright 436, Zombie Green, Texas Red, Rhodamine, and cyanine (Cy) dye (e.g., Cy2, Cy3, Cy5). Additional aspects provide methods of the disclosure where, (c) detecting occurs by immunoassay. The immunoassay utilized here can be selected from the group consisting of: lateral flow immunochromatography and/or enzyme-linked immunosorbent assay (ELISA), or more specifically, the immunoassay is selected from the group consisting of: enzyme immunoassay (EIA), ELISA, fluoroimmunoassay (FIA), chemiluminescent immunoassay (CLIA), counting immunoassay (CIA), and radioimmunoassay (RIA).

Additional embodiments of the disclosure provide for immunoassays that indicate the presence of at least one biomarker in a subject sample by a colorimetric change compared to a negative control sample, or the absence of at least one biomarker in a subject sample by a colorimetric change compared to a positive control sample. Further aspects can allow for quantification.

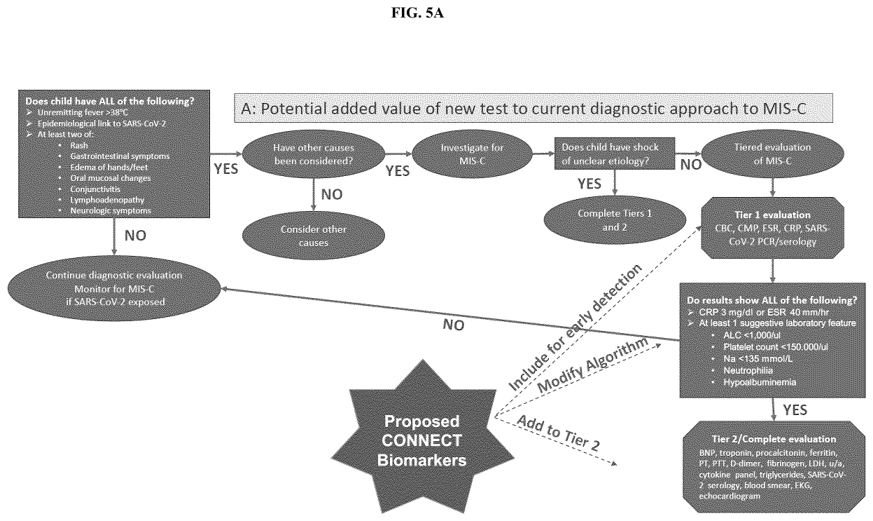

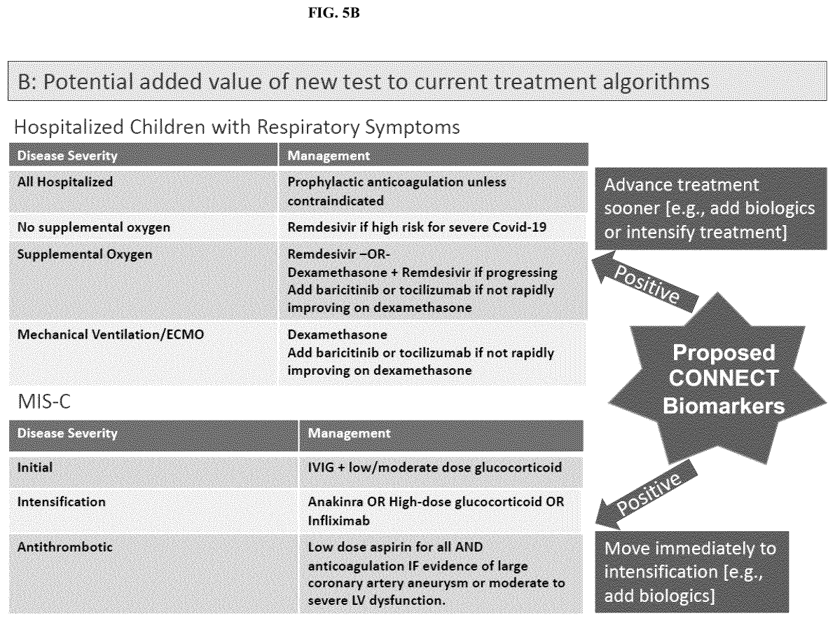

In some embodiments, the methods of the disclosure, further comprise: (i) measuring temperature of the subject (wherein the temperature is greater than (or equal to) 38° C.; (ii) identifying an epidemiological link to SARS-COV-2 infection; and (iii) identifying at least two of: (a) rash; (b) gastrointestinal symptoms; (c) edema of hands and/or feet; (d) oral mucosal changes; (c) conjunctivitis; (f) lymphoadenopathy; and (g) neurologic symptoms. Such methods also comprise evaluating the subject for other causes; investigating for MIS-C; identifying shock of an unclear etiology in the subject; or combinations thereof. Additional embodiments provide for such methods as described here, further comprising: (iv) measuring C-reactive protein (CRP); (v) measuring erythrocyte sedimentation rate (ESR); (vi) measuring complete blood count (CBC); (vii) measuring comprehensive metabolic panel (CMP); (viii) testing for SARS-COV-2 infection (e.g., PCR; antigen; serology); and (ix) measuring the at least one biomarker of a panel of biomarkers selected from the group consisting of Group (A) of TABLE 3 and/or Group (B) of TABLE 4, where if any one of the biomarkers of Group (A) of TABLE 3 is selected, then at least one biomarker of Group (B) of TABLE 4 is combined; or where the at least one biomarker comprises Macrophage mannose receptor 1/Mannose receptor C-type 1 (MRC1) and/or Tryptophan-tRNA ligase, cytoplasmic (WARS1), cither alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and/or any combinations thereof. In some aspects, the methods of the disclosure are directed to the at least one biomarker, where the at least one biomarker is selected from the group consisting of: FCGBP, MRC1, LBP, CD163, zonulin, and/or the at least one biomarker comprises a function selected from the group consisting of: mucosal immunity (e.g., mucosal immunity (e.g., mucins, Fc gamma binding protein (FCGBP), immunoglobulins (e.g., IgA, IgM), antimicrobial proteins (e.g., defensins, lysozyme), transferrins (e.g., lactoferrin)); macrophage activation (e.g., Mannose receptor C-type 1 (MRC1), CD163, CD14, Gal-3BP, matrix metalloproteinases (e.g., MMP7, MMP9), cytokines); bacterial infectivity (e.g., white blood cell count, erythrocyte sedimentation rate, procalcitonin, C-reactive protein (CRP), serum amyloid A (SAA), cytokines, lipopolysaccharide (LPS) binding protein (LBP)); intestinal damage/gut leakage (e.g., zonulin, fatty acid binding protein (FABP), glucagon-like peptide (GLP)-2, citrulline, lipopolysaccharide (LPS), LPS-binding protein (LBP)); any biomarker identified in TABLE 1: IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; and any combinations thereof; or where if the biomarker is at least one selected from the Group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the Group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof; or where the at least one biomarker comprises Macrophage mannose receptor 1/Mannose receptor C-type 1 (MRC1) and/or Tryptophan-tRNA ligase, cytoplasmic (WARS1), cither alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof; or where the at least one biomarker is selected from any one of Group (B) of TABLE 4, cither alone or in combination with any one of Group (A) of TABLE 3; and any combinations thereof. Some embodiments, for example, provide for biomarkers that are directed primarily to detection of MIS-C (see, e.g., TABLE 1, #1-19), while other biomarkers are directed to MIS-C and/or severe acute COVID-19 (see, e.g., TABLE 1, #20-23). Additional aspects of the method described here provide for (a) the C-reactive protein (CRP) level at 3 mg/dl or greater; (b) the erythrocyte sedimentation rate (ESR) is 40 mm/hr or greater; 20 mm/hr to 50 mm/hr (normal from men 0 to 22 mm/hr (≤15 mm/hr); normal women 0 to 29 mm/hr (≤20 mm/hr); normal child ≤10 mm/hr); (c) identifying at least one of: (1) absolute lymphocyte count of less than 1.000 μl; (2) platelet count of less than 150,000/μl; (3) sodium of less than 135 mmol/L; (4) neutrophilia; and (5) hypoalbuminemia; and (d) identifying the presence of at least one biomarker of a panel of biomarkers selected from the group consisting of: (1) a mucosal immunity biomarker (e.g., mucins, Fc gamma binding protein (FCGBP), zonulin, immunoglobulins (e.g., IgA, IgM), antimicrobial proteins (e.g., defensins, lysozyme), transferrins (e.g., lactoferrin)); (2) a macrophage activation biomarker (e.g., Mannose receptor C-type 1 (MRC1), CD163, CD14, Gal-3BP, matrix metalloproteinases (e.g., MMP7, MMP9), cytokines); and (3) a bacterial infectivity indicator (e.g., white blood cell count, erythrocyte sedimentation rate, procalcitonin, C-reactive protein (CRP), serum amyloid A (SAA), cytokines, lipopolysaccharide (LPS) binding protein (LBP)), and any combinations thereof; or at least one biomarker of a panel of biomarkers is selected from the group consisting of those in Group (A) of TABLE 3 and is in combination with at least one biomarker of Group (B) of TABLE 4, and any combinations thereof; or at least one biomarker of a panel of biomarkers is Macrophage mannose receptor 1/Mannose receptor C-type 1 (MRC1) and/or Tryptophan-tRNA ligase, cytoplasmic (WARS1) either alone or in combination with at least one biomarker selected from those in Group (A) of TABLE 3 and/or of Group (B) of TABLE 4, and any combinations thereof; or where the at least one biomarker is selected from any one of Group (B) of TABLE 4, either alone or in combination with any one of Group (A) of TABLE 3. See, e.g., FIG. 5A.

Further embodiments describe such methods also or further comprising at least one of: (x) measuring B-type natriuretic peptide (BNP); (xi) measuring troponin; (xii) measuring procalcitonin; (xiii) measuring ferritin; (xiv) measuring prothrombin time (PT); (xv) measuring partial thromboplastin time (PTT); (xvi) measuring D-dimer; (xvii) measuring fibrinogen; (xviii) measuring lactate dehydrogenase (LDH); (xix) measuring urinalysis (u/a); (xx) measuring cytokine panel; (xxi) measuring triglycerides; (xxii) measuring electrocardiogram (EKG); (xxiii) measuring echocardiogram; and (xxiv) testing blood smear; or any combinations thereof, where any one of these measurements are found to be abnormal or outside normal reference ranges (e.g., where the measurements are elevated compared to normal reference ranges). See, e.g., FIG. 5A. Some embodiments of the disclosure provide such methods, wherein the presence in a tested subject of at least one biomarker of (c) comprises a protein level of: at least a 2-fold change (e.g., 4, 6, 8, 10, 12, 14, 16, 18, 20); less than a 50-fold change (e.g., 49, 47, 45, 43, 41, 39, 37, 35, 33, 31, 29, 27, 25, 23, 21, 19, 17, 15, 13, 11, 9, 7, 5, 3); a 2-fold to a 50-fold change (e.g., 3-49; 4-48; 5-47; 6-46; 7-45; 8-44; 9-43; 10-42; 11-41; 12-40; 13-39; 14-38; 15-37; 16-36; 17-35; 18-34; 19-33; 20-32; 21-31; 22-30; 23-29; 24-28; 25-27) as compared to a protein level of a subject who does not suffer from MIS-C or severe acute COVID-19 (e.g., a healthy subject), or to a negative control indicative of the absence of any one of the at least one biomarkers described here. For example, a subject sample can comprise any one or more of the biomarkers described here (TABLES 1-4), where the biomarker is elevated or decreased when compared to normal reference ranges of a control sample and/or a sample from a healthy subject lacking MIS-C and/or severe acute COVID-19. Non-limiting examples of serum biomarker protein levels in a tested subject, where the serum biomarker protein level is indicative of the subject suffering from MIS-C and/or severe acute COVID-19, include: MRC1: 0.2 mg/L or greater (e.g., 0.4, 0.6, 0.8, 2, 4, 6, 8, 10, 12, 14, 16, 18, 20); 21 mg/L or less (e.g., 19, 17, 15, 13, 11, 9, 7, 5, 3, 1, 0.9, 0.7, 0.5, 0.3, 0.1); 0.2 mg/L-21 mg/L (e.g., 0.3-20, 0.4-19, 0.5-18, 0.4-17, 0.5-16, 0.6-15, 0.7-14, 0.8-13, 0.9-12, 1-11, 2-10, 3-9, 4-8, 5-7); LBP: 3 mg/L or greater (e.g., 5, 7, 9, 11, 13, 15, 17, 19, 21, 23, 25, 27, 29, 31, 33, 35, 37, 39, 41); 155 mg/L or less (e.g., 154, 152, 150, 148, 146, 144, 142, 140, 138, 136, 134, 132, 130, 128, 126, 124, 122, 120, 118, 116, 114, 112, 110, 108, 106, 104, 102, 100, 98, 96, 94, 92, 90, 88, 86, 84, 82, 80, 78, 76, 74, 72, 70, 68, 66, 64, 62, 60, 58, 56, 54, 52, 50, 48, 46, 44, 42, 40, 38, 36, 34, 32, 30, 28, 26, 24, 22, 20, 18, 16, 14, 12, 10, 8, 6, 4, 2); 3 mg/L-155 mg/L (e.g., 4-154, 5-153, 6-152, 7-151, 8-150, 9-149, 10-148, 11-147, 12-146, 13-145, 14-144, 15-143, 16-142, 17-141, 18-140, 19-139, 20-138, 21-137, 22-136, 23-135, 24-134, 25-133, 24-132, 25-131, 26-130, 27-129, 28-128, 29-127, 30-126, 31-125, 32-124, 33-123, 34-122, 35-121, 36-120, 37-119, 38-118, 39-117, 40-116, 41-115, 42-114, 43-113, 44-112, 45-111, 46-110, 47-109, 48-108, 49-107, 50-106, 51-105, 52-104, 53-103, 54-102, 55-101, 56-100, 57-99, 58-98, 59-97, 60-96, 61-95, 62-94, 63-93, 64-92, 65-91, 66-90, 67-89, 68-88, 69-87, 70-86, 71-85, 72-84, 73-83, 74-82, 75-81. 76-80, 77-79). In additional embodiments, methods of the disclosure are provided, wherein at least one biomarker is selected from the group consisting of: wherein at least one biomarker is selected from the group consisting of: mucosal immunity biomarkers (e.g., mucins, Fc gamma binding protein (FCGBP), immunoglobulins (e.g., IgA, IgM), antimicrobial proteins (e.g., defensins, lysozyme), transferrins (e.g., lactoferrin)); Mannose receptor C-type 1 (MRC1), CD163, CD14, Gal-3BP, matrix metalloproteinases (e.g., MMP7, MMP9), cytokines; white blood cell count, erythrocyte sedimentation rate, procalcitonin, C-reactive protein (CRP), serum amyloid A (SAA), cytokines, lipopolysaccharide (LPS) binding protein (LBP); and combinations thereof, and any combinations thereof; or at least one biomarker of a panel of biomarkers is selected from the group consisting of those in Group (A) of TABLE 3 and is in combination with at least one biomarker of Group (B) of TABLE 4, and any combinations thereof; or at least one biomarker of a panel of biomarkers is Macrophage mannose receptor 1/Mannose receptor C-type 1 (MRC1) and/or Tryptophan-tRNA ligase, cytoplasmic (WARS1) either alone or in combination with at least one biomarker selected from those in Group (A) of TABLE 3 and/or of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)), and any combinations thereof; or where the at least one biomarker is selected from any one of Group (B) of TABLE 4, either alone or in combination with any one of Group (A) of TABLE 3.

Some embodiments are directed to any of the described methods, where at least one capture antibody is configured to detect or is against at least one biomarker for diagnosing and/or prognosing Multisystem Inflammatory Syndrome in Children (MIS-C) or mild or asymptomatic SARS-COV-2 infection disclosed in TABLE 5.

In further embodiments, any of the methods described here include at least one capture antibody that is configured to detect or is against at least one biomarker for diagnosing and/or prognosing MIS-C or severe COVID-19 disclosed in TABLE 6.

In additional embodiments described here, the methods of diagnosing and/or prognosing further comprise (d) treating comprises: (i) administering at least one of: immunoglobulins (e.g., intravenous 2 g/kg (maximum of 100 g); IgG primarily without or with lower amounts of: IgA, IgD, IgE, IgM; PRIVIGEN®: 99.2% IgG, 0.8% monomers and dimers with IgA (1.8 mg/ml); GAMUNEX®-C: IgG/10% Caprylate/Chromatography Purified; intravenous: 300-600 mg/kg (1 mg/kg/min-8 mg/kg/min); subcutaneous: 1.37× current IV dose in g/IV dose interval in weeks; 10 mL/hr/site-15 mL/hr/site; 10 mL/hr/site-20 mL/hr/site per week; OCTAGAM® 5% liquid: 96% IgG. 50 mg IgG/ml; percent of total IgG: IgG1 is 65%, IgG2 is 30%, IgG3 is 3% and IgG4 is 2%)); steroids (e.g., intravenous 2-30 mg/kg/day methylprednisolone); immunomodulators or biologics (e.g., (e.g., anakinra 2-10 mg/kg/day via subcutaneous or intravenous, divided every 6 hrs to 12 hrs)); antibiotics; cytokine inhibitors; and any combination thereof in an effective amount sufficient to: reduce, alleviate, or slow the progression of a symptom of MIS-C, wherein the symptom of MIS-C is selected from the group consisting of: fever that lasts 24 hours or longer; vomiting; diarrhea; stomach pain; skin rash; fatigue; inability to wake up or stay awake; fast heartbeat; rapid breathing; difficulty breathing; red eyes; redness or swelling of the lips and tongue or hands or feet; pale lips or nail beds; headache, dizziness or lightheadedness; confusion; enlarged lymph nodes; and any combinations thereof. Some aspects of the methods described here provide for treating a subject suffering from MIS-C or severe acute COVID-19, wherein administering comprises: oral administration, intravenous administration, intramuscular administration, or combinations thereof. In some embodiments, the methods of detecting at least one biomarker and/or diagnosing MIS-C, or severe acute COVID-19 in a subject suspected from suffering MIS-C, or severe acute COVID-19 as described here can be used in combination with or without the methods of treating a subject suspected of or suffering from MIS-C, or severe acute COVID-19. The methods of detecting and the methods of treatment when used in combination accelerate the diagnosis and treatment, and improves prognosis of the subject.

Some embodiments provide methods described here for administering a treatment, where the treatment comprises a combination and administering the combination occurs: together, separately, simultaneously (essentially), sequentially (essentially), or combinations thereof. In additional embodiments described here, the methods are useful, wherein the subject is a human, such as but not limited to, a child, a teenager, a human less than 21 years old. Furthermore, the methods of diagnosing and/or prognosing a subject suspected of suffering from MIS-C or severe acute COVID-19, which utilizes an apparatus, wherein the apparatus (wherein the apparatus is configured for capture antibody coating, deposition, or conjugation) is selected from the group consisting of: a membrane (e.g., nitrocellulose; polyvinylidene fluoride (PVDF)); a multi-well plate (e.g., polystyrene, polyvinyl chloride); a bead; a chip (e.g., a microfluidic chip or a microchip); and a slide (e.g., glass) (wherein the apparatus is configured for capture antibody coating, deposition, or conjugation).

In some embodiments, the method described here utilizes a sample from a human subject, such as but not limited to a child, a teen, a subject of less than 21 years of age, where the method requires only a small volume of sample from the subject, for example, 5 microliters (μl) or greater (e.g., 7 μl, 9 μl, 11 μl, 13 μl, 15 μl, 17 μl, 19 μl, 21 μl, 23 μl, 25 μl, 50 μl, 75 μl, 125 μl, 175 μl, 225 μl, 275 μl, 325 μl, 375 μl, 425 μl, 475 μl, 525 μl, 575 μl, 625 μl, 675 μl, 725 μl, 775 μl, 825 μl, 875 μl, 925 μl, 975 μl, 1025 μl); 1 milliliter (ml) or less (e.g., 0.9 ml, 0.8 ml, 0.7 ml, 0.6 ml, 0.5 ml, 0.4 ml, 0.3 ml, 0.2 ml, 0.1 ml, 0.09 ml, 0.08 ml, 0.07 ml, 0.06 ml, 0.05 ml, 0.04 ml, 0.03 ml, 0.02 ml, 0.01 ml, 0.009 ml, 0.008 ml, 0.007 ml, 0.006 ml, 0.005 ml); 5 μl to 1 ml (e.g., 6 μl-950 μl; 7 μl-850 μl; 8 μl-750 μl; 9 μl-650 μl; 10 μl-550 μl; 11 μl; -450 μl; 12 μl-350 μl; 13 μl-250 μl; 14 μl-150 μl; 15 μl-50 μl; 16 μl-48 μl; 17 μl-46 μl; 18 μl; -44 μl; 19 μl-42 μl; 20 μl-40 μl; 21 μl-38 μl; 22 μl-36 μl; 23 μl-34 μl; 24 μl-32 μl; 25 μl-30 μl), where the sample can be a blood sample (e.g., serum, plasma), or any other contemplated sample that contains at least one biomarker for MIS-C and/or severe acute COVID-19 from a subject. The method described here, in some embodiments, can be directed to administering an immunoglobulin treatment, where the immunoglobulin comprises a majority of: immunoglobulins, such as but not limited to, IgG1, IgG2, IgG3, IgG4, or combinations thereof, and none or a minority of: IgA (e.g., IgA1, IgA2), IgM, IgD, IgE, and any combination thereof. One embodiment of the method can comprise a blood sample from a subject suspected of suffering from MIS-C or severe acute COVID-19, where the blood sample comprises at least one biomarker of MIS-C or severe acute COVID-19).

Some embodiments providing the method of diagnosing a subject suffering from MIS-C or severe acute COVID-19 quickly and efficiently as described here, where the method further comprises treating the subject containing at least one biomarker indicative of MIS-C or severe acute COVID-19, where the treatment comprises administration of a therapeutic (e.g., immunoglobulins (e.g., intravenous 2 g/kg (maximum of 100 g); IgG primarily without or with lower amounts of: IgA, IgD, IgE, IgM; PRIVIGEN®: 99.2% IgG, 0.8% monomers and dimers with IgA (1.8 mg/ml); GAMUNEX®-C: IgG/10% Caprylate/Chromatography Purified; intravenous: 300-600 mg/kg (1 mg/kg/min-8 mg/kg/min); subcutaneous: 1.37× current IV dose in g/IV dose interval in weeks; 10 mL/hr/site-15 mL/hr/site; 10 mL/hr/site-20 mL/hr/site per week; OCTAGAM® 5% liquid: 96% IgG, 50 mg IgG/ml; percents of total IgG: IgG1 is 65%, IgG2 is 30%, IgG3 is 3% and IgG4 is 2%)); steroids (e.g., intravenous 2-30 mg/kg/day methylprednisolone); immunomodulators or biologics (e.g., (e.g., anakinra 2-10 mg/kg/day via subcutaneous or intravenous, divided every 6 hrs to 12 hrs)); antibiotics; cytokine inhibitors) in an amount sufficient to be treat, reduce, alleviate, slow the progression of, or combinations thereof, at least one symptom of MIS-C or severe acute COVID-19. See, e.g., FIGS. 5A-5B. The methods described here provide for sensitive and early detection of MIS-C and/or severe acute COVID-19 in a subject (e.g., child, teen, adult less than 21 years old).

Additional embodiments of methods of treatment of the disclosure comprise treating. reducing, alleviating, slowing the progression of, or combinations thereof, MIS-C or severe acute COVID-19 or at least one symptom thereof, in a subject suspected of or suffering from MIS-C or severe acute COVID-19. In some embodiments, the method described here comprises administering at least one therapeutic selected from the group consisting of: intravenous immunoglobulin (e.g., 2 g/kg IBW/dose (up to a maximum total dose of 100 g) IV for 1 dose; 1 g/kg IBW/dose IV every 24 hours for 2 doses, if cardiac dysfunction or fluid overload); steroid (e.g., methylprednisolone 1-2 mg/kg/dose IV every 12 hours; if non-responsive to 1-2 mg/kg/dose IV every 12 hours, increase to 10-30 mg/kg/day (up to a maximum of 1,000 mg/day) IV for 1 to 2 days); immunomodulator or biologic (e.g., anakinra (5-10 mg/kg/day IV or subcutaneous 1-3 divided doses); infliximab (e.g., 5-10 mg/kg/dose IV for 1 dose); enoxaparin (e.g., >2 months to <18 years: prophylaxis 0.5 mg/kg/dose (up to a maximum of 30 mg/dose) subcutaneous every 12 hours; >2 months to <18 years: treatment 1 mg/kg/dose subcutaneous every 12 hours; antifactory Xa activity (treatment goal 0.5 to 1)); aspirin (e.g., 3-5 mg/kg/dose (up to maximum of 81 mg/dose) per oral once daily). Positive results for an elevated presence of at least one of the biomarkers described here indicate that a treatment plan should be advanced and/or intensified. For example, subjects with respiratory symptoms that are: (1) hospitalized, then prophylactic anticoagulation treatments unless contraindicated; (2) without supplemental oxygen, then remdesivir if high risk for severe acute COVID-19; (3) on supplemental oxygen, then remdesivir or dexamethasone and remdesivir if progressing, add baricitinib or tocilizumab if not rapidly improving on dexamethasone; (4) on mechanical ventilation/ECMO, then dexamethasone, or add baricitinib or tocilizumab if not rapidly improving on dexamethasone, should be administered. For those subjects diagnosed with MIS-C with a disease severity of: (1) initial stages, then IVIG and low/moderate dose of glucocorticoid; (2) intensification, then anakinra or high-dose glucocorticoid or infliximab; (3) anti-thrombotic, then low dose aspirin for all and anticoagulation if evidence of large coronary artery aneurysm or moderate to severe left ventricle (LV) dysfunction, should be administered. See, e.g., FIGS. 5A-5B. In some embodiments, methods of treatment described here are performed by oral administration, intravenous administration, subcutaneous administration, intramuscular administration, or combinations thereof. Additional embodiments provide for a treatment cycle comprising administering (e.g., orally, intravenously, subcutaneously) at least one therapeutic (e.g., 2, 3, 4, 5) for one or more times per day (e.g., 2, 3, 4, 5, 6). In further embodiments, the treatment cycle comprises administering for at least 1 day consecutively or alternating days ((e.g., 2, 3, 4, 5, 6, 7, 8, 9, 10, 11, 12, 13, 14); 21 consecutive days or fewer (e.g., 20, 19, 18, 17, 16, 15, 14, 13, 12, 11, 10, 9, 8, 7, 6, 5, 4, 3, 2, 1); or 1-21 consecutive days (e.g., 2-20; 3-19; 4-18; 5-17; 6-16; 7-15; 8-14; 9-13; 10-12). Some embodiments of the disclosure provide methods of treating, where the at least one therapeutic in a treatment regimen disclosed here comprises, consists essentially of, or consists of at least two therapeutics (e.g., 3, 4, 5, 6), where the at least two therapeutics can be administered together, separately, simultaneously or essentially simultaneously, sequentially or essentially sequentially, or any combinations thereof. Additional embodiments of the methods of treating described here comprises administering the at least one therapeutic 1 time, 2 times, 3 times, or 4 times per day; administering the at least one therapeutic once every 24 hours, 12 hours, 8 hours, or 6 hours; or administering the at least one therapeutic 4 times per day at once every 6 hours. In some embodiments, methods of treating the subject diagnosed with MIS-C, or severe acute COVID-19 by administering at least one therapeutic in an effective amount sufficient to: treat MIS-C, or severe acute COVID-19, reduce or alleviate the at least one symptom of MIS-C, or severe acute COVID-19, slow the progression of MIS-C, or severe acute COVID-19, induce remission, or combinations thereof; and further comprising subjecting the subject to an additional standard of care treatment addressing any one or more of: persistent fever, inflammation, organ dysfunction or shock; gastrointestinal symptoms (e.g., abdominal pain, diarrhea, nausea/vomiting); toxic shock syndrome-like features with hemodynamic instability and poor heart function; cytokine storm/macrophage activation or hyperinflammatory features; thrombosis or acute kidney injury; congestive heart failure or pulmonary embolism; respiratory symptoms; Kawasaki disease-like features (e.g., coronary artery enlargement; aneurysms; swollen glands; neurologic manifestations; rash; conjunctivitis, red eyes; red or swollen hands and feet) and combinations thereof. The methods of detecting and/or diagnosing and treating MIS-C, or severe acute COVID-19, or symptoms of any of the forgoing, described here comprise a subject that is an animal or a mammal, for example, a human, or any mammal suspected of or suffering from MIS-C, or severe acute COVID-19.

Further embodiments, incorporating various elements described here, such as but not limited to, the route of administration, the effective amounts, the dosage regimen, and the like, provide methods of preventing, inhibiting, or decreasing development and/or progression of MIS-C, or severe acute COVID-19, in a subject diagnosed with MIS-C, or severe acute COVID-19 using the methods described here. The methods of preventing, inhibiting, or decreasing development and/or progression of MIS-C, or severe acute COVID-19 in the subject, comprising administering at least one of the therapeutics described here in an amount effective to treat reduce, alleviate, slow the progression of, or combinations thereof, MIS-C, or severe acute COVID-19, thereby treating the subject suffering from MIS-C, or severe acute COVID-19. In additional embodiments, these methods of treatment can be used in combination with or without the methods of detecting described here.

### Apparatus

Additional embodiments provide an apparatus as described here, where the apparatus, comprises: at least one capture antibody attached thereto, wherein at least one capture antibody is configured to detect or is against at least one biomarker for diagnosing and/or prognosing Multisystem Inflammatory Syndrome in Children (MIS-C) or severe acute COVID-19, wherein at least one biomarker comprises a function selected from the group consisting of: mucosal immunity (sec, e.g., R. Bansil and B.S. Turner. Adv Drug Deliv Rev. 2018 Jan. 15; 124:3-15; P. Brandtzaeg. Scand J Immunol. 2009 December; 70(6):505-15.; J.M. Crecth. Br Med Bull. 1978 January; 34(1):17-24) (e.g., mucins, Fc gamma binding protein (FCGBP), immunoglobulins (e.g., IgA, IgM), antimicrobial proteins (e.g., defensins, lysozyme), transferrins (e.g., lactoferrin)); macrophage activation (Moin et al. Sci Rep. 2021 Mar. 19; 11(1):6428; Shaked et al. Arterioscler Thromb Vase Biol. 2014 May; 34(5):1085-92) (e.g., Mannose receptor C-type 1 (MRC1), CD163, CD14, Gal-3BP, matrix metalloproteinases (e.g., MMP7, MMP9), cytokines); bacterial infectivity (e.g., white blood cell count, erythrocyte sedimentation rate, procalcitonin (see, e.g., Vijayan et al. J Intensive Care. 2017 Aug. 3; 5:51), C-reactive protein (CRP), serum amyloid A (SAA), cytokines, lipopolysaccharide (LPS) binding protein (LBP)); intestinal damage/gut leakage (e.g., zonulin, FABP, GLP-2, citrulline, LPS, LBP); any biomarker identified in TABLE 1: IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; and any combinations thereof; or where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Further embodiments provide for such methods as described here, where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Other embodiments are directed to such methods described here where the at least one biomarker is selected from any one of Group (B) of TABLE 4, either alone or in combination with any one of Group (A) of TABLE 3, and any combinations thereof. Some embodiments provide for biomarkers that are directed primarily to detection of MIS-C (see, e.g., TABLE 1, #1-19), while other biomarkers are directed to MIS-C and/or severe acute COVID-19 (see, e.g., TABLE 1, #20-23). The apparatus, in some embodiments, can comprise a panel of capture antibodies against any of the at least one biomarker described here. Aspects directed to such apparatuses are selected from the group consisting of: a membrane, a multi-well plate, a bead, a chip, and a slide. In some aspects, the apparatus described here comprises at least one biomarker selected from the group consisting of: mucosal immunity (e.g., mucins, Fc gamma binding protein (FCGBP), immunoglobulins (e.g., IgA, IgM), antimicrobial proteins (e.g., defensins, lysozyme), transferrins (e.g., lactoferrin)); Mannose receptor C-type 1 (MRC1), CD163, CD14, Gal-3BP, matrix metalloproteinases (e.g., MMP7, MMP9), cytokines; white blood cell count, erythrocyte sedimentation rate, procalcitonin, C-reactive protein (CRP), serum amyloid A (SAA), cytokines, lipopolysaccharide (LPS) binding protein (LBP); intestinal damage/gut leakage (e.g., zonulin, fatty acid binding protein (FABP), glucagon-like peptide (GLP)-2, citrulline, lipopolysaccharide (LPS), LPS-binding protein (LBP)); any biomarker identified in TABLE 1: IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; and any combinations thereof. Some aspects describe such apparatuses comprising a panel of biomarkers, where the at least one biomarker comprises (consists essentially of or consists of): FCGBP; MRC1; LBP; CD163 (or FCGBP; MRC1; and LBP); IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or any combinations thereof; where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Further embodiments provide for such methods as described here, where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Other embodiments are directed to such methods described here where the at least one biomarker is selected from any one of Group (B) of TABLE 4, either alone or in combination with any one of Group (A) of TABLE 3. In further aspects, the apparatus disclosed here comprises at least one capture antibody, where the antibody is against the at least one biomarker, and specifically, the at least one capture antibody is selected from the group consisting of: an antibody against FCGBP; an antibody against MRC1; an antibody against LBP; an antibody against CD163; an antibody against zonulin; and combinations thereof. In some aspects, the at least one capture antibody is selected from the group consisting of: a monoclonal antibody against FCGBP; a monoclonal antibody against MRC1; a monoclonal antibody against LBP; a monoclonal antibody against CD163; a monoclonal antibody against IgGFc-binding protein/Fc gamma binding protein; a monoclonal antibody against Apolipoprotein C-III; a monoclonal antibody against Nidogen-1; a monoclonal antibody against Macrophage mannose receptor 1/Mannose receptor C-type 1; a monoclonal antibody against Cathepsin D; an antibody against Vascular cell adhesion protein 1; a monoclonal antibody against Junction plakoglobin; a monoclonal antibody against Proteasome subunit alpha type-5; a monoclonal antibody against Methanethiol oxidase; a monoclonal antibody against Ferritin light chain; a monoclonal antibody against Endoplasmin; a monoclonal antibody against Proteasome subunit alpha type-1; a monoclonal antibody against Sulfhydryl oxidase 1; a monoclonal antibody against Proteasome subunit beta type-1; a monoclonal antibody against Tryptophan-tRNA ligase, cytoplasmic; a monoclonal antibody against Plasma kallikrein; a monoclonal antibody against Desmocollin-1; a monoclonal antibody against Alpha-1-antichymotrypsin; a monoclonal antibody against Thrombospondin-4; a monoclonal antibody against Prenylcysteine oxidase; a monoclonal antibody against Insulin-like growth factor binding protein 3; a monoclonal antibody against Insulin-like growth factor binding protein complex, acid labile subunit; a monoclonal antibody against Inter-alpha-trypsin inhibitor heavy chain H2; a monoclonal antibody against any one of the biomarker proteins identified in any one of TABLES 1-6; and any combinations thereof; or where the at least one monoclonal antibody is against a biomarker, where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the monoclonal antibody of the at least one biomarker of TABLE 3 is combined with a monoclonal antibody against at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count. erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Further embodiments provide for such apparatuses as described here comprising at least one monoclonal antibody, where the monoclonal antibody is against at least one biomarker of: Macrophage mannose receptor 1/Mannose receptor C-type 1 (MRC1) and/or Tryptophan-tRNA ligase, cytoplasmic (WARS1), either alone or in combination with a monoclonal antibody against at least one biomarker (or at least one biomarker comprising a function) selected from the group consisting of: a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Other embodiments are directed to such apparatuses described here comprising at least one monoclonal antibody against the at least one biomarker selected from any one of Group (B) of TABLE 4, either alone or in combination with any one of Group (A) of TABLE 3; and/or any combinations thereof.

Some embodiments provide an apparatus as described here, where the apparatus comprises at least one capture antibody configured to detect or is against at least one biomarker for diagnosing and/or prognosing Multisystem Inflammatory Syndrome in Children (MIS-C) or mild or asymptomatic SARS-COV-2 infection disclosed in TABLE 5.

In further embodiments, an apparatus as described here provides at least one capture antibody that is configured to detect or is against at least one biomarker for diagnosing and/or prognosing MIS-C or severe COVID-19 disclosed in TABLE 6.

Additional aspects provide for the apparatus described here, wherein the apparatus (wherein the apparatus is configured for capture antibody coating, deposition, or conjugation) is selected from the group consisting of: a membrane (e.g., nitrocellulose; polyvinylidene fluoride (PVDF)); a multi-well plate (e.g., polystyrene, polyvinyl chloride); a bead; a chip (e.g., a microfluidic chip or a microchip); and a slide (e.g., glass) (wherein the apparatus is configured for capture antibody coating, deposition, or conjugation).

Non-limiting examples of detectable labels for use in the methods and/or apparatus described here, where the detectable labels include: horseradish peroxidase (HRP), alkaline phosphatase (AP), β-galactosidase, glucose oxidase, glucose-6-phosphate dehydrogenase, β-N-acetylglucosaminidase, invertase, xanthine oxidase, biotin, Fluorescein Isothiocyanate (FITC), Tetramethylrhodamine-5-(and 6)-isothiocyanate (TRITC), Alexa Fluor® (AF) dye (e.g., AF 350, AF 405, AF 430, AF 488, AF 500, AF 514, AF 532, AF 546, AF 555, AF 568, AF 594, AF 610, AF 633, AF 635, AF 647, AF 660, AF 680, AF 700, AF 750, AF 790), Allophycocyanin (APC), phycoerythrin (PE), Brilliant violet (BV) dye (e.g., BV 421, BV 480, BV 510, BV 570, BV 605, BV 650, BV 711, BV 750, BV 785), Peridinin-chlorophyll-protein (PerCP), Pacific blue, Pacific orange, PE/Dazzle 594, APC Fire 750, Super Bright 436, Zombie Green, Texas Red, Rhodamine, and cyanine (Cy) dye (e.g., Cy2, Cy3, Cy5). In some embodiments, the method further comprises detecting the detection antibody, where the detection antibody reflects the presence of at least one biomarker in the subject. For example, the detection occurs by an immunoassay. Non-limiting immunoassays include lateral flow immunochromatography, enzyme-linked immunosorbent assay (ELISA), enzyme immunoassay (EIA), fluoroimmunoassay (FIA), chemiluminescent immunoassay (CLIA), counting immunoassay (CIA), and radioimmunoassay (RIA). A person of ordinary skill in the art understands an appropriate detection antibody for use with the immunoassay of choice.

In some embodiments, the methods of the disclosure comprise an apparatus or the use of an apparatus. One embodiment provides for the apparatus described here, where the apparatus can be selected from: a membrane or a multi-well plate, where the membrane comprises a nitrocellulose membrane or a polyvinylidene fluoride (PVDF) membrane, or the multi-well plate comprises a polystyrene multi-well plate or polyvinyl chloride multi-well plate, or a bead (e.g., a magnetic bead), or a chip (e.g., a microfluidic chip or microchip), or a slide, where the apparatus is configured to enable or allow immobilization, coating, protein deposition, or conjugation of at least one capturing antibody or a plurality of capturing antibodies. In embodiments of the disclosure, the apparatuses described here comprise at least one capturing antibody, or a plurality of capturing antibodies, which binds an antigen or an antigen conjugate, or pluralities thereof. Additional exemplary representative, but non-limiting antigens or antigen conjugates (e.g., biomarkers of MIS-C, or severe acute COVID-19) are provided here.

Some embodiments of the disclosure are directed to the apparatus described here for use in an immunoassay, such as but not limited to, lateral flow immunochromatography or enzyme-linked immunosorbent assay (ELISA) for diagnosing a subject with MIS-C, or severe acute COVID-19.

Additional embodiments are directed to an apparatus comprising at least one capturing antibody configured to bind at least one antigen or biomarker described here, or a plurality of biomarker antigens, which can be used in any of the disclosed embodiments for diagnosing MIS-C, or severe acute COVID-19. In some embodiments, at least one conjugated detection antibody (antibody conjugated with a detectable label) binds at least one biomarker antigen.

### Definitions

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By the term “consist essentially of”, “consists essentially of”, or “consisting essentially of”, it is meant that the components or ingredients include only the listed components along with the normal impurities present in commercial materials and with any other additives present at levels which do not affect the operation of embodiments of the disclosure, for instance at levels less than 5% by weight or less than 1% or even 0.5% by weight.

The term “effective amount” or “therapeutically effective amount” of an agent (e.g compounds having the structure of formula (I), etc.), as used herein, is that amount sufficient to effect beneficial or desired results, such as clinical results, and, as such, an “effective amount” depends upon the context in which it is being applied. In some embodiments, the therapeutics are administered in an effective amount for the treatment or prophylaxis of a disease, disorder or condition, such as for example, MIS-C, or severe acute COVID-19. In another embodiment, in the context of administering an agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., cancer, etc.); and remission (whether partial or total), whether detectable or undetectable, as compared to the response obtained without administration of the agent.

Determining an effective amount of substance to be delivered can depend upon a number of factors including. for example, the chemical or biologic structure and biological activity of the substance, the age and weight of the subject, the precise condition requiring treatment and its severity, the treatment schedule or duration, and the route of administration. The term “administer” or “administration” includes the delivery of a composition or one or more pharmaceutically active ingredients to an animal, individual, or subject, such as a human, including, for example, any suitable methods that serve to deliver the composition or its active ingredients or other pharmaceutically active ingredients to the site in need of treatment. The route of administration can vary depending on various factors, such as, for example, the components or ingredients of the pharmaceutical composition or the type or nature of the pharmaceutically active and/or inert ingredients, the location in need of treatment, for example, the site of the potential or actual infection, the causative agent of the disease or disorder, e.g., microorganism involved in the disease or disorder, the severity of the disease or disorder, the age and physical condition of the individual, and the like, which a person of ordinary skill would understand to take into consideration. Some non-limiting examples of routes of administration of a composition or pharmaceutically active ingredient to an individual include oral (e.g., solid (e.g., tablet, capsule, caplet, powder), gel (e.g., gel cap), solution (e.g., syrup, drink)); intravenous (e.g., as a sterile solution free of particulate emboli and in a solvent system suitable for intravenous use); topical (e.g., as a cream, gel, lotion, or ointment); intrapulmonary, intraperitoneal, intramuscular, parenteral, sublingual, transdermal, intranasal, inhalation (e.g., nebulizer, spray), intraocular, intratracheal, intrarectal, vaginal, genicular skin, ocular (e.g., eye drops), ear drops, and the like. In the case of a pharmaceutical composition that contains more than one ingredient (active or inert), one way of administering such a composition is by mixing the ingredients (for example, in the form of a suitable unit dosage form, such as a tablet, capsule, solution, powder, etc.) and then the introduction of the dosage form. The ingredients can also be administered separately (simultaneously or sequentially, i.e., one after the other), provided that these ingredients reach favorable therapeutic levels so that the composition as a whole has a synergistic and/or desired effect.

The frequency of treatments depends upon a number of factors, such as the amount of the therapeutic composition administered per dose, the pharmacodynamics and pharmacokinetics of the therapeutic agent(s), as well as the health and history of the subject. The precise amount, number of doses, and timing of doses will be determined by the attending physician or veterinarian. Therapeutic compositions of the present disclosure can be administered to any subject or animal, including various mammals.

As used herein, the term “subject” refers to any organism to which a composition and/or compound in accordance with the disclosure may be administered, e.g., for experimental, diagnostic, prophylactic, and/or therapeutic purposes. Non-limiting examples of typical subjects or individuals include any animal (e.g., mammals such as dogs, cats, mice, rats, rabbits, sheep, pigs, horses, cows, cattle, non-human primates, and humans, etc.). A subject in need thereof is typically a subject for whom it is desirable to treat a disease, disorder, or condition as described herein. For example, a subject in need thereof can seek or be in need of treatment, require treatment, be receiving treatment, receive treatment in the future, or a human or animal that is under care by a trained professional for a particular disease, disorder, or condition. In some embodiments described here, the subject is a human, a pediatric individual, a child, a teenager, or a human less than 21 years old for diagnosing and/or prognosing and/or treating for MIS-C and/or severe acute COVID-19.

Typically, the treatment of a disease, disorder, or condition (e.g., the conditions described herein such as MIS-C, or severe acute COVID-19) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### EXAMPLES

The following examples illustrate specific aspects of the instant description. The examples should not be construed as limiting, as the example merely provides specific understanding and practice of the embodiments and its various aspects.

### Example 1: Proteomics Analysis

A semiquantitative detection of the biomarkers in plasma as diagnostic and prognostic markers in children suspected to have Multisystem Inflammatory Syndrome in Children (MIS-C) or severe acute COVID-19 at the time of emergency evaluation is designed. Each of these biomarkers or proteins are detectable and elevated in plasma during sepsis or organ damage in various severe diseases. Biomarker levels, either in combination or by themselves, can be proportional to disease severity. These biochemical indicators of organ damage are diagnostic and therapeutic biomarkers for MIS-C and/or severe, acute SARS-COV-2-induced illness. For example, high plasma levels of FCGBP, MRCI (e.g., 0.43 mg/L-6.2 mg/L; Rødgaard-Hansen, et al. Clin Chem Lab Med. 52(3):453-461, 2014), and LBP (e.g., ˜46.2 mg/L; Zweigner et al. Blood. 98:3800-3808, 2001) can be associated with organ damage in children with MIS-C, particularly the gut, prior to clinical manifestations, where testing for these biomarkers increase the accuracy and/or speed of MIS-C diagnosis and intervention and lead to better outcomes. From a therapeutic viewpoint, elevated plasma levels of any of the biomarkers are used as early indicators of a non-response to standard treatments and enable faster definitive therapeutic decisions.

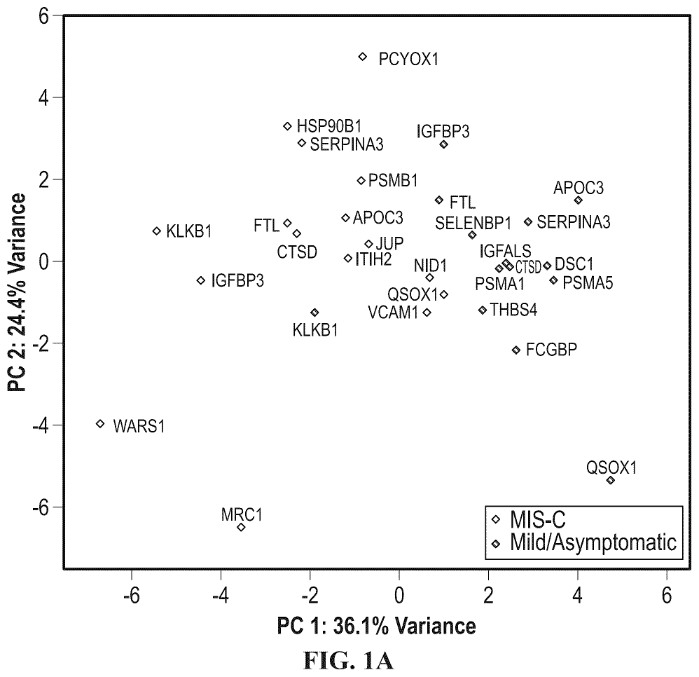

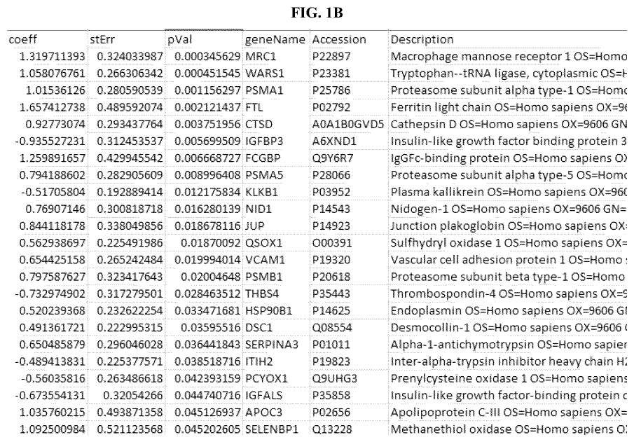

A principal components analysis was performed to show how to distinguish pediatric subjects with MIS-C from those who had experienced a mild/asymptomatic SARS-COV-2 infection based on the 23 protein biomarkers listed in TABLE 1. The data were log2-transformed using y=log₂(x+0.02) and then the principal components were computed. Positive values of y indicated overexpression in MIS-C patients compared to patients who had experienced a mild/asymptomatic SARS-COV-2 infection. FIG. 1 illustrates a plot of the first two principal components with the two groups (MIS-C and mild/asymptomatic) coded as red and blue, respectively. The MIS-C (red) datapoints were primarily found below 0 on the X-axis and between −2 to +2 on the Y-axis (with outliers at (X, Y): (−3.5, −6.5); (−6.5, −4); (−2, +3)), while the mild/asymptomatic COVID-19 (blue) datapoints were primarily found between 0 to +4 on the X-axis and −2 to +2 on the Y-axis (with outliers at (X, Y): (+4.5, −5.5); (−1, +4.5); (+1, +2.5)),. The separation showed that the 23 proteins in TABLE 1 can distinguish between patient subjects with MIS-C from those who had experienced a mild/asymptomatic SARS-COV-2 infection.

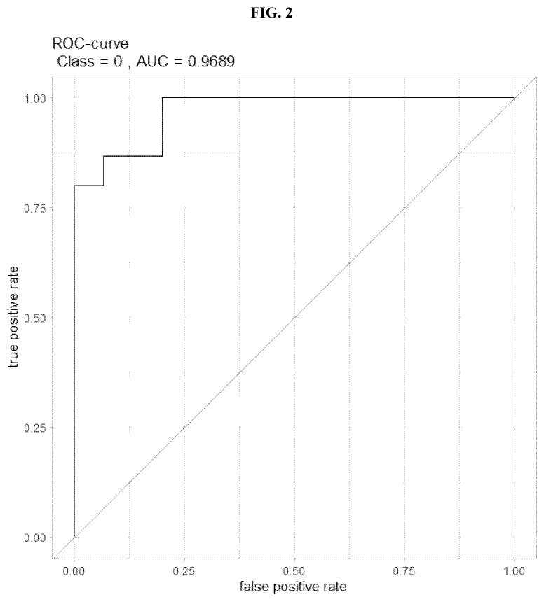

A predictive model for MIS-C was estimated using abundance levels of the 23 proteins listed in TABLE 1 using a machine learning method called “support vector machines” to predict the classes of patient subjects, which are either “MIS-C” or “mild/asymptomatic”. The prediction algorithm gave a misclassification rate of 0.133. FIG. 2 shows the corresponding receiver operating characteristic (ROC) curve. The diagonal line is a null reference with an area under the curve of 0.5. Values of the Area Under the Curve (AUC) greater than 0.5 indicate a model that is effective at making predictions across a range of threshold values. with an area of 1.0 indicating a perfect predictive model. The support vector machine model based on the 23 proteins described here has an AUC=0.9689, which demonstrates a high degree of classification accuracy.

### Example 2: Assay Development

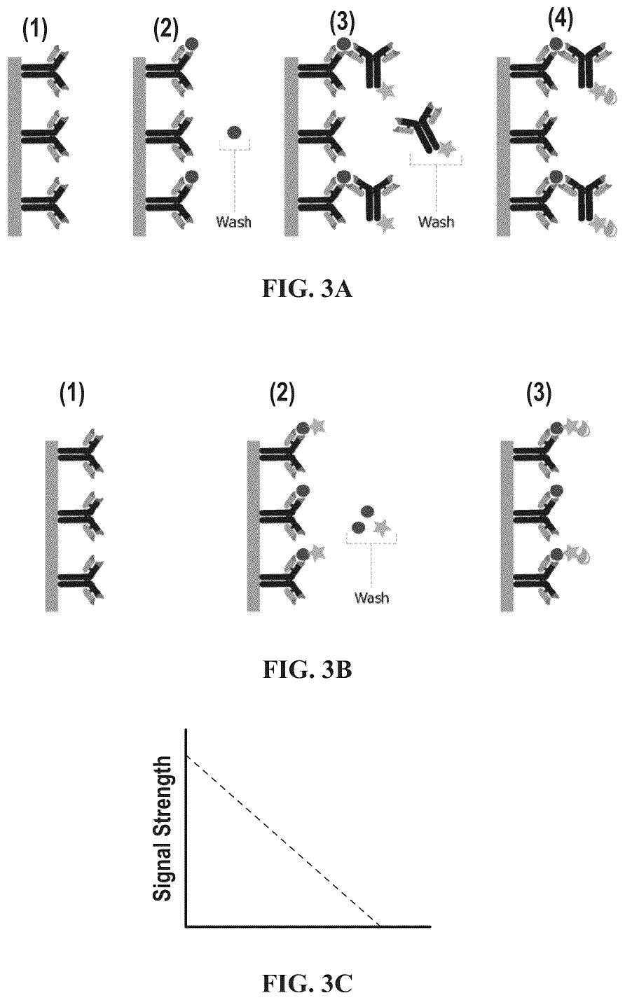

An in-house sandwich ELISA utilizing standard protocols is illustrated in FIG. 3A. The key reagents for this assay are pairs of capture and detection antibodies, with which binding of one antibody to the test antigen is not inhibited by binding of the other antibody. Capture and detection monoclonal antibody pairs against any of the biomarkers, such as those that are commercially available for human MRC1 (e.g., human MRC1 capture antibody, ab281180 (recombinant anti-mannose receptor antibody, BSA-and azide-free); human MRC1 detector antibody, ab281030; abcam; Waltham, MA, USA) and human LBP (e.g., human lipopolysaccharide-binding protein (LBP) capture antibody (unconjugated) (recombinant anti-LBP antibody, BSA-and azide-free), ab281528; human LBP detector antibody (unconjugated), ab2815520; abcam; Waltham, MA, USA). Other sources also exist and can be used. Monoclonal antibodies against human Fc Fragment of IgG Binding Protein (FCGBP) also commercially available (e.g., abx172355, Abbexa, Inc.; MAP389Hu21, Cloud-clone Corp.; Katy, TX, USA), which are tested as capture and detection pair.

If a capture and detection pair is not appropriate for sandwich ELISA, a competitive ELISA can utilize, e.g., purified recombinant FCGBP (Novus Biologicals, mybiosource.com, and others) that are biotinylated utilizing standard protocols; detection by HRP-streptavidin is illustrated in FIG. 3B). HRP conjugation or biotinylation of detection antibodies for sandwich ELISA can be conducted utilizing commercial reagents and standard protocols. Matrix equivalency studies can be performed to compare assay results in plasma versus serum from the same individual subjects. Commercially available (for research only) sandwich ELISA kits for the detection of FCGBP, MRC1, and LBP can be used for comparison. FIG. 3C shows that signal strength (Y-axis) is inversely proportional to the concentration of the analyte (X-axis) in competitive ELISA or other competitive immunoassays.

### Example 3: Biomarker Validation

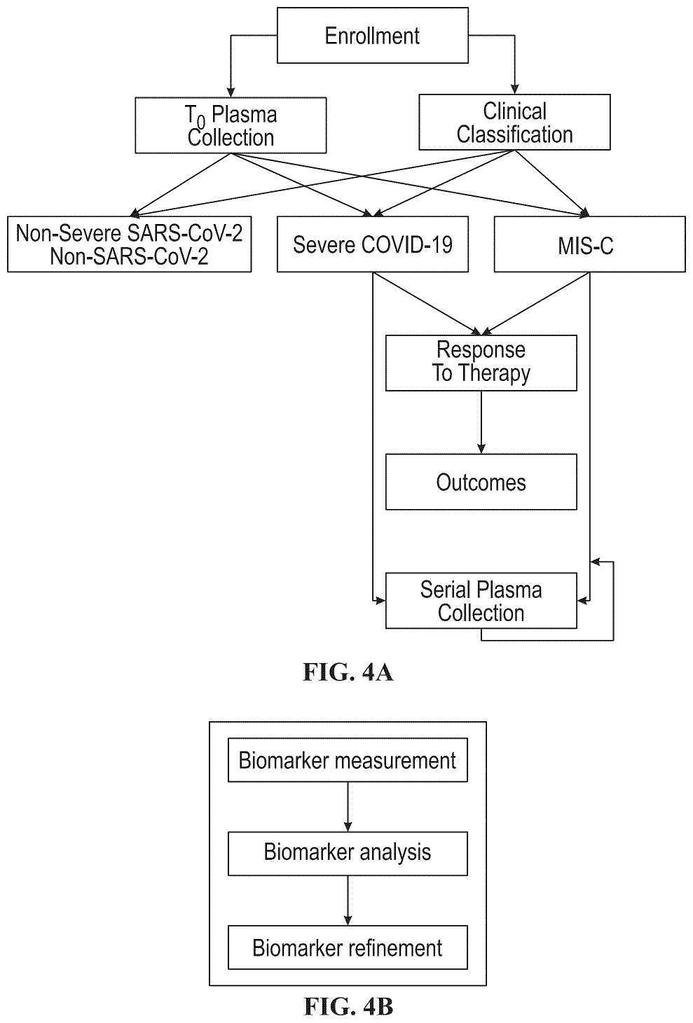

A prospective cohort study and nested case-control study are performed within the CONNECT network. Children and adolescents presenting with suspected severe acute COVID-19 or MIS-C at the Emergency Department (ED) of participating network sites are recruited and consented, and plasma are collected at the time of initial blood draw (FIG. 4A). Results of plasma biomarker assays are compared between participants hospitalized for severe acute COVID-19 or MIS-C (cases) and a random subset of controls, which include those: a) discharged home from the ED with COVID-19; b) discharged home from the ED with a final diagnosis other than severe acute COVID-19 or MIS-C; and c) hospitalized with final diagnosis other than severe acute COVID-19 or MIS-C. Electronic health record data are collected on clinical course and response to therapy, including antiviral medication for severe acute COVID-19 or intravenous immunoglobulin (IVIG) and glucocorticoids for MIS-C. Cases are sampled serially during hospitalization, and at 1, 3, and 6 months after clinical presentation. Follow-up data are collected at post-discharge timepoints via brief online questionnaires or telephone interviews.

As summarized in FIG. 4B (inset), measurements of biomarker for cases and controls are performed in a blinded fashion. Biomarker level cutoff values are initially based on preliminary results. Biomarker levels are associated with clinical diagnosis and outcomes. Cutoffs wareere iteratively refined based on the accuracy of prediction. Additionally, biomarker levels are incorporated as predictor variables into larger predictive models incorporating sociodemographic and clinical characteristics.

### Example 4: Alternative Approaches

The added value of the bioassay to the existing battery of clinical laboratory tests was the early (preclinical) identification of organ damage, particularly of the gut, which would guide diagnosis of MIS-C and therapeutic decisions. In severe COVID-19, biomarker detection complements or precedes the results of blood tests for pulmonary function.

Pediatric samples from the acutely infected group were used in biomarker validation. Since the targets for validation were relatively small (<50 per group), and the required sample volume was <50 μl, these alternative approaches enabled these targets. Similar alternative approaches, including use of disease-specific biorepositories, were used for control samples.

A correlation between prolonged symptoms and persistently high biomarker levels was found. The biomarkers can contribute to the understanding of post-acute sequelae of SARS-COV-2 infection (PASC), since altered biomarker levels would be easily accessible indicators of persisting organ damage. Moreover, if viral persistence was found to underlie (at least some) PASC manifestations, biomarkers of organ damage were ideal surrogate markers of viral persistence, since they were amenable to immunoassay formats commonly used in clinical laboratory settings. This contrasted with the direct detection of viral products, which required ultrasensitive detection methods (e.g., single molecule arrays) and expensive, frequently unavailable instrumentation.

### Example 5: Determination of Biomarkers

Exemplary biomarkers identified in TABLE 1 were obtained by analyzing plasma from subjects suffering from MIS-C. severe COVID-19, or mild/asymptomatic SARS-COV-2 infection. The samples were analyzed through unbiased quantitative proteomics using isobaric labeling protein microchemistry and mass spectrometry, followed by statistical analysis. The analysis utilized two comparisons: (1) MIS-C incident vs Mild/asymptomatic; and (2) Severe acute COVID-19 incident vs Mild/asymptomatic. The protein biomarkers were selected for those that exhibited log2 differences between groups (1) and (2) that were >0.4 and: (a) were significantly different (p<0.05) in MIS-C incident vs Mild/asymptomatic, but not in Severe acute COVID-19 incident vs Mild/asymptomatic; or (b) in MIS-C vs Mild/asymptomatic and in Severe acute COVID-19 incident vs Mild/asymptomatic. Those protein biomarkers that were identified as exhibiting a log2 difference of >0.4 between groups (1) and (2) and had a statistically significant difference in MIS-C incident vs mild/asymptomatic, but not in severe acute COVID-19 incident vs mild/asymptomatic are proteins #1-19 in TABLE 1. Biomarker proteins #20-23 in TABLE 1 were identified as those that exhibited a log2 difference of >0.4 between groups (1) and (2) and had a statistically significant difference in MIS-C incident vs mild/asymptomatic and in severe COVID-19 incident vs mild/asymptomatic. All of the proteins identified in TABLE 1 exhibited a False Discovery Rate ≤ 10%. TABLE 2 provides a subclassification of biomarker and one factor in diagnosing MIS-C and/or severe acute COVID-19.

### Example 6: Determination of Biomarkers from MIS-C and Control Subjects

Exemplary biomarkers identified in TABLE 5 were obtained by analyzing plasma samples from 17 MIS-C patients and 20 control subjects with a history of mild or asymptomatic SARS-COV-2 infection or COVID-19. Samples were analyzed by unbiased data independent acquisition (DIA) mass spectrometry. From a total of 1801 proteins. 769 proteins were selected that had fewer than half the values missing in both groups. For these 769 proteins, analysis of the log2-transformed value representing relative abundance of the proteins was used. These values were analyzed via a linear model that is equivalent to a Student's t-test. To correct for multiple testing. the p-values for all 769 comparisons were transformed to experiment-wise significance levels via the Holm's method. The list of 34 proteins of TABLE 5 was selected based on experiment-wise (Holm-corrected for multiple comparisons) p-values less than 0.05.

### Example 7: Determination of Biomarkers from MIS-C and Severe COVID-19 Subjects

Exemplary biomarkers identified in TABLE 6 were obtained by analyzing plasma samples from 17 MIS-C patients and 20 severe COVID-19 patients. Samples were analyzed by unbiased data independent acquisition (DIA) mass spectrometry. From a total of 1801 proteins, 676 proteins were selected that had fewer than half the values missing in both groups. For these 676 proteins, analysis of the log2-transformed value representing relative abundance of the proteins was used. These values were analyzed via a linear model that is equivalent to a Student's t-test. To correct for multiple testing, the p-values for all comparisons were transformed to q-values using the Benjamini-Hochberg procedure. The list of 15 proteins of TABLE 6 was selected based on q-values lower than 0.05, corresponding to a false discovery rate of 0.05.

TABLE 1

| | | | PROTEIN |
|---|---|---|---|
| NO. | PROTEIN | GENE | ACCESSION NO. |
| 1 | IgGFc-binding protein/ | FCGBP | Q9Y6R7 |
| | Fc gamma binding protein | | |
| 2 | Apolipoprotein C-III | APOC3 | P02656 |
| 3 | Nidogen-1 | NID1 | P14543 |
| 4 | Macrophage mannose | MRC1 | P22897 |
| | receptor 1/Mannose | | |
| | receptor C-type 1 | | |
| 5 | Cathepsin D | CTSD | A0A1B0GVD5 |
| 6 | Vascular cell adhesion | VCAM1 | P19320 |
| | protein 1 | | |
| 7 | Junction plakoglobin | JUP | P14923 |
| 8 | Proteasome subunit | PSMA5 | P28066 |
| | alpha type-5 | | |
| 9 | Methanethiol oxidase | SELENBP1 | Q13228 |
| 10 | Ferritin light chain | FTL | P02792 |
| 11 | Endoplasmin | HSP90B1 | P14625 |
| 12 | Proteasome subunit | PSMA1 | P25786 |
| | alpha type-1 | | |
| 13 | Sulfhydryl oxidase 1 | QSOX1 | O00391 |
| 14 | Proteasome subunit | PSMB1 | P20618 |
| | beta type-1 | | |
| 15 | Tyrptophan--tRNA ligase, | WARS1 | P23381 |
| | cytoplasmic | | |
| 16 | Plasma kallikrein | KLKB1 | P03952 |
| 17 | Desmocollin-1 | DSC1 | Q08554 |
| 18 | Alpha-1-antichymotrypsin | SERPINA3 | P01011 |
| 19 | Thrombospondin-4 | THBS4 | P35443 |
| 20 | Prenylcysteine oxidase | PCYOX1 | Q9UHG3 |
| 21 | Insulin-like growth factor | IGFBP3 | P17936 |
| | binding protein 3 | | |
| 22 | Insulin-like growth factor | IGFALS | P35858 |
| | binding protein complex, | | |
| | acid | | |
| 23 | Inter-alpha-trypsin | ITIH2 | P19823 |
| | inhibitor heavy chain H2 | | |

TABLE 2

| NO. | PROTEIN | GENE | TYPE |
|---|---|---|---|
| 1 | IgGFc-binding protein/ | FCGBP | MIS-C |
| | Fc gamma binding protein | | |
| 2 | Apolipoprotein C-III | APOC3 | MIS-C |
| 3 | Nidogen-1 | NID1 | MIS-C |
| 4 | Macrophage mannose | MRC1 | MIS-C |
| | receptor 1/Mannose receptor | | |
| | C-type 1 | | |
| 5 | Cathepsin D | CTSD | MIS-C |
| 6 | Vascular cell adhesion | VCAM1 | MIS-C |
| | protein 1 | | |
| 7 | Junction plakoglobin | JUP | MIS-C |
| 8 | Proteasome subunit alpha | PSMA5 | MIS-C |
| | type-5 | | |
| 9 | Methanethiol oxidase | SELENBP1 | MIS-C |
| 10 | Ferritin light chain | FTL | MIS-C |
| 11 | Endoplasmin | HSP90B1 | MIS-C |
| 12 | Proteasome subunit alpha | PSMA1 | MIS-C |
| | type-1 | | |
| 13 | Sulhydryl oxidase 1 | QSOX1 | MIS-C |
| 14 | Proteasome subunit beta | PSMB1 | MIS-C |
| | type-1 | | |
| 15 | Tryptophan--tRNA ligase, | WARS1 | MIS-C |
| | cytoplasmic | | |
| 16 | Plasma kallikrein | KLKB1 | MIS-C |
| 17 | Desmocollin-1 | DSC1 | MIS-C |
| 18 | Alpha-1-antichymotrypsin | SERPINA3 | MIS-C |
| 19 | Thrombospondin-4 | THBS4 | MIS-C |
| 20 | Prenylcysteine oxidase | PCYOX1 | MIS-C; |
| | | | severe acute |
| | | | COVID-19 |
| 21 | Insulin-like growth factor | IGFBP3 | MIS-C; |
| | binding protein 3 | | severe acute |
| | | | COVID-19 |
| 22 | Insulin-like growth factor | IGFALS | MIS-C; |
| | binding protein complex, | | severe acute |
| | acid labile subunit | | COVID-19 |
| 23 | Inter-alpha-trypsin | ITIH2 | MIS-C; |
| | inhibitor heavy chain H2 | | severe acute |
| | | | COVID-19 |

TABLE 3
GROUP (A)

| PROTEIN | GENE |
|---|---|
| IgGFc-binding protein/ | FCGBP |
| Fc gamma binding protein | |
| Alpha-1-antichymotrypsin | SERPINA3 |
| Cathepsin D (CTSD); Ferritin light chain | FTL |
| Proteasome subunit alpha type-1 | PSMA1 |
| Sulfhydryl oxidase 1 | QSOX1 |
| Nidogen-1 | NID1 |
| Apolipoprotein C-III | APOC3 |
| insulin-like growth factor binding protein 3 | IGFBP3 |
| insulin-like growth factor binding protein | IGFALS |
| complex, acid labile subunit | |
| Plasma kallikrein | KLKB1 |
| Inter-alpha-trypsin inhibitor heavy chain H2 | ITIH2 |

TABLE 4
GROUP (B)

| PROTEIN BIOMARKER | GENE |
|---|---|
| Macrophage mannose receptor 1/ | MRC1 |
| Mannose receptor C-type 1 | |
| Tryptophan--tRNA ligase, cytoplasmic | WARS1 |
| Vascular cell adhesion protein 1 | VCAM1 |
| junction plakoglobin | JUP |
| Proteasome subunit alpha type-5 | PSMA5 |
| Methanethiol oxidase | SELENBP1 |
| Ferritin light chain | FTL |
| Endoplasmin | HSP90B1 |
| Proteasome subunit beta type-1 | PSMB1 |
| Desmocollin-1 | DSC1 |
| Thrombospondin-4 | THBS4 |
| Prenylcysteine oxidase | PCYOX1 |
| mucosal immunity (e.g., mucosal immunity (e.g., mucins, | |
| immunoglobulins (e.g., IgM), antimicrobial proteins (e.g., | |
| defensins, lysozyme), transferrins (e.g., lactoferrin)) | |
| macrophage activation (e.g., | |
| Mannose receptor C-type 1 (MRC1), Gal-3BP, | |
| matrix metalloproteinases (e.g., MMP7, MMP9)) | |
| bacterial infectivity (e.g., | |
| white blood cell count, erythrocyte sedimentation | |
| rate, procalcitonin, serum amyloid A (SAA), | |
| intestinal damage/gut leakage (e.g., fatty acid | |
| binding protein (FABP), glucagon-like peptide | |
| (GLP)-2, citrulline, lipopolysaccharide (LPS)) | |

TABLE 5

| PROTEIN BIOMARKER | GENE | FUNCTION |
|---|---|---|
| Serum amyloid A-1 protein | SAA1 | Heparin binding |
| Inter-alpha-trypsin inhibitor | ITIH1 | Serine protease inhibitor |
| heavy chain H1 | | |
| Alpha-2-HS-glycoprotein | AHSG | Endopeptidase inhibitor |
| Attractin | ATRN | Immune cell clustering |
| | | during inflammation |
| Histidine-rich glycoprotein | HRG | Endopeptidase inhibitor |
| von Willebrand factor | VWF | Hemostasis maintenance |
| Alpha-1-antichymotrypsin | SERPINA3 | Serine protease inhibitor |
| Gelsolin | GSN | Actin modulator |
| Inter-alpha-trypsin inhibitor | ITIH2 | Serine protease inhibitor |
| heavy chain H2 | | |
| Scavenger receptor cysteine- | CD163 | Endocytosis of hemoglobin/ |
| rich type 1 protein M130 | | haptoglobin complexes |
| Serotransferrin | TF | Ferrous iron transport |
| Alpha-1-acid glycoprotein 1 | ORM1 | Transport protein |
| C-reactive protein | CRP | Host defense modulator |
| Phosphatidylinositol-glycan- | GPLD1 | Hydrolase |
| specific phospholipase D | | |
| IgGFc-binding protein | FCGBP | Mucosal layer integrity |
| Glutathione peroxidase 3 | GPX3 | Peroxidase |
| Tenascin | TNC | Cell-matrix adhesion |
| Cartilage intermediate layer | CILP2 | Cartilage extracellular |
| protein 2 | | matrix scaffolding |
| Fetuin-B | FETUB | Endopeptidase inhibitor |
| Vascular cell adhesion protein | VCAM1 | Leukocyte-endothelium |
| 1 | | adhesion |
| Peptidase inhibitor 16 | PI16 | Peptidase inhibitor |
| Macrophage | MRC1 | Endocytosis of glycoproteins, |
| mannose receptor1 | | pathogen phagocytosis |
| Low affinity immunoglobulin | FCGR3A | Antigen-IgG complez binding |
| gamma Fc region | | |
| receptor III-A | | |
| Lumican | LUM | Cell-matrix adhesion |
| Zinc-alpha-2-glycoprotein | AZGP1 | Stimulates lipid degradation |
| Extracellular matrix protein 1 | ECM1 | Extracellular matrix |
| | | structural component |
| Contactin-1 | CNTN1 | Cell adhesion |
| Insulin-like growth factor 2 | IGF2 | Growth factor |
| Serum | PON1 | Hydrolase |
| paraoxonase/arylesterase 1 | | |
| Insulin-like growth factor- | IGFALS | Insulin-like growth |
| binding protein complex acid | | factor binding |
| labile subunit | | |
| Alpha-2-macroglobulin | A2M | Protease inhibitor |
| Cholinesterase | BCHE | Hydrolase |
| Corticosteroid-binding | SERPINA6 | Steroid binding/Serine |
| globulin | | protease inhibitor |
| Insulin-like growth factor- | IGFBP3 | Insulin-like growth |
| binding protein 3 | | factor binding |

TABLE 6

| PROTEIN BIOMARKER | GENE | FUNCTION |
|---|---|---|
| Gamma-glutamyl hydrolase | GGH | Hydrolase |
| Uncharacterized protein | C18orf63 | Unknown |
| C18orf63 | | |
| Thyroxine-binding globulin | SERPINA7 | Serine protease inhibitor |
| Macrophage mannose | MRC1 | Endocytosis of glycoproteins, |
| receptor 1 | | pathogen phagocytosis |
| Type 2 lactosamine alpha- | ST3GAL6 | Sialyltransferase, |
| 2,3-sialyltransferase | | glycolipid biosynthesis |
| Complement component C8 | C8B | Complement activation |
| beta chain | | |
| Lymphatic vessel endothelial | LYVE1 | Transmembrane |
| hyaluronic acid receptor 1 | | signaling receptor |
| Calicin | CCIN | Spermiogenic differentiation |
| Macrophage colony- | CSF1R | Tyrosine-protein kinase |
| stimulating factor 1 receptor | | |
| Zinc-alpha-2-glycoprotein | AZGP1 | Stimulates lipid degradation |
| Alpha-1- | AMBP | Oxidoreductase |
| microglobulin/Bikunin | | |
| precursor | | |
| IgGFc-binding protein | FCGBP | Mucosal layer integrity |
| Galectin-3-binding protein | LGALS3BP | Cell adhesion |
| Adenosine deaminase 2 | ADA2 | Hydrolase |
| Properdin | CFP | Complement activation |

### Specific Embodiments

Non-limiting specific embodiments are described below, each of which is considered to be within the present disclosure.

Embodiment 1. An apparatus, comprising:
at least one capture antibody attached thereto,
   wherein the at least one capture antibody is configured to detect at least one biomarker for diagnosing and/or prognosing Multisystem Inflammatory Syndrome in Children (MIS-C) or severe acute COVID-19,
      wherein the at least one biomarker is selected from the group consisting of: at least one biomarker comprising a function selected from the group consisting of: mucosal immunity (e.g., mucins, Fc gamma binding protein (FCGBP), immunoglobulins (e.g., IgA, IgM), antimicrobial proteins (e.g., defensins, lysozyme), transferrins (e.g., lactoferrin)); macrophage activation (e.g., Mannose receptor C-type 1 (MRC1), CD163, CD14, Gal-3BP, matrix metalloproteinases (e.g., MMP7, MMP9), cytokines); bacterial infectivity (e.g., white blood cell count, erythrocyte sedimentation rate, procalcitonin, C-reactive protein (CRP), serum amyloid A (SAA), cytokines, lipopolysaccharide (LPS) binding protein (LBP)); intestinal damage/gut leakage (e.g., fatty acid binding protein (FABP), glucagon-like peptide (GLP)-2, citrulline, lipopolysaccharide (LPS), LPS-binding protein (LBP)); IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker is selected from any one of Group (B) of TABLE 4, either alone or in combination with any one of Group (A) of TABLE 3; and any combinations thereof.

Embodiment 2. The apparatus of embodiment 1, wherein the apparatus is selected from the group consisting of: a membrane, a multi-well plate, a bead, a chip, and a slide.

Embodiment 3. The apparatus of embodiment 1, wherein the at least one biomarker is selected from the group consisting of: mucosal immunity (e.g., mucins, Fc gamma binding protein (FCGBP), immunoglobulins (e.g., IgA, IgM), antimicrobial proteins (e.g., defensins, lysozyme), transferrins (e.g., lactoferrin)); Mannose receptor C-type 1 (MRC1), CD163, CD14, Gal-3BP, matrix metalloproteinases (e.g., MMP7, MMP9), cytokines; white blood cell count, erythrocyte sedimentation rate, procalcitonin, C-reactive protein (CRP), serum amyloid A (SAA), cytokines, lipopolysaccharide (LPS) binding protein (LBP); and any combinations thereof.

Embodiment 4. The apparatus of embodiment 1, wherein the at least one biomarker comprises (consists essentially of or consists of): FCGBP; MRC1; LBP; CD163; IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; (or FCGBP; MRC1; and LBP); or where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker is selected from any one of Group (B) of TABLE 4, cither alone or in combination with any one of Group (A) of TABLE 3; or any combination thereof.

Embodiment 5. The apparatus of embodiment 3, wherein the at least one capture antibody is an antibody against the at least one biomarker.

Embodiment 6. The apparatus of embodiment 1, wherein the at least one capture antibody is selected from the group consisting of: an antibody against FCGBP; an antibody against MRC1; an antibody against LBP; an antibody against CD163; an antibody against IgGFc-binding protein/Fe gamma binding protein; an antibody against Apolipoprotein C-III; an antibody against Nidogen-1; an antibody against Macrophage mannose receptor 1/Mannose receptor C-type 1; an antibody against Cathepsin D; an antibody against Vascular cell adhesion protein 1; an antibody against Junction plakoglobin; an antibody against Proteasome subunit alpha type-5; an antibody against Methanethiol oxidase; an antibody against Ferritin light chain; an antibody against Endoplasmin; an antibody against Proteasome subunit alpha type-1; an antibody against Sulfhydryl oxidase 1; an antibody against Proteasome subunit beta type-1; an antibody against Tryptophan-tRNA ligase, cytoplasmic; an antibody against Plasma kallikrein; an antibody against Desmocollin-1; an antibody against Alpha-1-antichymotrypsin; an antibody against Thrombospondin-4; an antibody against Prenylcysteine oxidase; an antibody against Insulin-like growth factor binding protein 3; an antibody against Insulin-like growth factor binding protein complex, acid labile subunit; an antibody against Inter-alpha-trypsin inhibitor heavy chain H2; antibody against any one of the proteins identified in any one of TABLES 1-4; and any combinations thereof; or where the at least one antibody is against a biomarker, where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the antibody of the at least one biomarker of TABLE 3 is combined with an antibody against at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof.

Embodiment 7, The apparatus of claim Embodiment 1, wherein the at least one capture antibody is against at least one biomarker of: Macrophage mannose receptor 1/Mannose receptor C-type 1 (MRC1) and/or Tryptophan-tRNA ligase, cytoplasmic (WARS1), either alone or in combination with an antibody against at least one biomarker (or at least one biomarker comprising a function) selected from the group consisting of: a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or
wherein the at least one capture antibody is against the at least one biomarker selected from any one of Group (B) of TABLE 4, either alone or in combination with any one of Group (A) of TABLE 3; and any combinations thereof.

Embodiment 8. The apparatus of embodiment 1, wherein the at least one capture antibody is selected from the group consisting of: a monoclonal antibody against FCGBP; a monoclonal antibody against MRC1; a monoclonal antibody against LBP; a monoclonal antibody against CD163; a monoclonal antibody against IgGFc-binding protein/Fc gamma binding protein; a monoclonal antibody against Apolipoprotein C-III; a monoclonal antibody against Nidogen-1; a monoclonal antibody against Macrophage mannose receptor 1/Mannose receptor C-type 1; a monoclonal antibody against Cathepsin D; an antibody against Vascular cell adhesion protein 1; a monoclonal antibody against Junction plakoglobin; a monoclonal antibody against Proteasome subunit alpha type-5; a monoclonal antibody against Methanethiol oxidase; a monoclonal antibody against Ferritin light chain; a monoclonal antibody against Endoplasmin; an antibody against Proteasome subunit alpha type-1; a monoclonal antibody against Sulfhydryl oxidase 1; a monoclonal antibody against Proteasome subunit beta type-1; a monoclonal antibody against Tryptophan-tRNA ligase, cytoplasmic; a monoclonal antibody against Plasma kallikrein; a monoclonal antibody against Desmocollin-1; a monoclonal antibody against Alpha-1-antichymotrypsin; a monoclonal antibody against Thrombospondin-4; a monoclonal antibody against Prenylcysteine oxidase; a monoclonal antibody against Insulin-like growth factor binding protein 3; a monoclonal antibody against Insulin-like growth factor binding protein complex, acid labile subunit; a monoclonal antibody against Inter-alpha-trypsin inhibitor heavy chain H2; or
a monoclonal antibody against any one of the biomarker proteins identified in any one of TABLES 1-4;
where the at least one monoclonal antibody is against a biomarker,
   where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); insulin-like growth factor binding protein complex, acid labile subunit (IGFALS); Plasma kallikrein (KLKB1); Inter-alpha-trypsin inhibitor heavy chain H2 (ITIH2),
      then the monoclonal antibody of the at least one biomarker of TABLE 3 is combined with a monoclonal antibody against at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof. Further embodiments provide for such apparatuses as described here comprising at least one monoclonal antibody, where the monoclonal antibody is against at least one biomarker of: Macrophage mannose receptor 1/Mannose receptor C-type 1 (MRC1) and/or Tryptophan-tRNA ligase, cytoplasmic (WARS1), either alone or in combination with a monoclonal antibody against at least one biomarker (or at least one biomarker comprising a function) selected from the group consisting of: a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or at least one monoclonal antibody against the at least one biomarker selected from any one of Group (B) of TABLE 4, either alone or in combination with any one of Group (A) of TABLE 3; and any combinations thereof.

Embodiment 9. The apparatus of embodiment 1, wherein the apparatus (wherein the apparatus is configured for capture antibody coating, deposition, or conjugation) is selected from the group consisting of: a membrane (e.g., nitrocellulose; polyvinylidene fluoride (PVDF)); a multi-well plate (e.g., polystyrene, polyvinyl chloride); a bead; a chip (e.g., a microfluidic chip or a microchip); and a slide (e.g., glass) (wherein the apparatus is configured for capture antibody coating, deposition, or conjugation).

Embodiment 10. A method, comprising:
(a) contacting at least one capture antibody and a sample (e.g., blood; plasma; serum) from a subject suspected of or suffering from Multisystem Inflammatory Syndrome in Children (MIS-C) or severe acute COVID-19, thereby forming a contacted sample on an apparatus (as described in embodiments 1-8);
(b) binding the contacted sample and a detection entity that binds to at least one biomarker of the sample;
(c) detecting the detection entity,
   wherein the detection entity reflects or indicates the presence of at least one biomarker for Multisystem Inflammatory Syndrome in Children (MIS-C) or severe acute COVID-19 in the subject; and
(d) treating the subject for Multisystem Inflammatory Syndrome in Children (MIS-C) or severe acute COVID-19,
   wherein the at least one biomarker comprises a function selected from the group consisting of: mucosal immunity (e.g., mucosal immunity (e.g., mucins, Fc gamma binding protein (FCGBP), immunoglobulins (e.g., IgA, IgM), antimicrobial proteins (e.g., defensins, lysozyme), transferrins (e.g., lactoferrin)); macrophage activation (e.g., Mannose receptor C-type 1 (MRC1), CD163, CD14, Gal-3BP, matrix metalloproteinases (e.g., MMP7, MMP9), cytokines); bacterial infectivity (e.g., white blood cell count, erythrocyte sedimentation rate, procalcitonin, C-reactive protein (CRP), serum amyloid A (SAA), cytokines, lipopolysaccharide (LPS) binding protein (LBP)); intestinal damage/gut leakage (e.g., fatty acid binding protein (FABP), glucagon-like peptide (GLP)-2, citrulline, lipopolysaccharide (LPS), LPS-binding protein (LBP)); IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or the at least one biomarker is selected from any one of Group (B) of TABLE 4, either alone or in combination with any one of Group (A) of TABLE 3; and any combinations thereof.

Embodiment 11. The method of embodiment 10, wherein the apparatus comprises the at least one capture antibody,
wherein the at least one capture antibody is configured to detect the at least one biomarker for diagnosing and/or prognosing Multisystem Inflammatory Syndrome in Children (MIS-C) or severe acute COVID-19.

Embodiment 12. The method of embodiment 10, wherein the sample comprises at least one biomarker for Multisystem Inflammatory Syndrome in Children (MIS-C) or severe acute COVID-19 in the subject.

Embodiment 13. The method of embodiment 10, further comprising reducing background interference (e.g., blocking; washing) on the apparatus.

Embodiment 14. The method of embodiment 13, wherein reducing background interference occurs by washing the apparatus comprising the contacted sample after (a) contacting at least one capture antibody and the sample (e.g., blood; plasma; serum) from the subject suspected of or suffering from Multisystem Inflammatory Syndrome in Children (MIS-C) or severe acute COVID-19; after (b) binding the contacted sample and the detection entity that binds to at least one biomarker; or both before and after (b) binding the contacted sample and the detection entity that binds to at least one biomarker.

Embodiment 15. The method of embodiment 10, wherein the detection entity comprises specificity for the at least one biomarker.

Embodiment 16. The method of embodiment 10, wherein the detection entity comprises specificity for the at least one capture antibody.

Embodiment 17. The method of embodiment 10, wherein the detection entity comprises a detectable label.

Embodiment 18. The method of embodiment 17, wherein the detectable label is selected from the group consisting of: an enzyme label, a fluorescent label, and a biotin label.

Embodiment 19. The method of embodiment 17 or 18, wherein the detectable label is selected from the group consisting of: horseradish peroxidase (HRP), alkaline phosphatase (AP), β-galactosidase, glucose oxidase, glucose-6-phosphate dehydrogenase, β-N-acetylglucosaminidase, invertase, xanthine oxidase, biotin, Fluorescein Isothiocyanate (FITC), Tetramethylrhodamine-5-(and 6)-isothiocyanate (TRITC), Alexa Fluor® (AF) dye (e.g., AF 350, AF 405, AF 430, AF 488, AF 500, AF 514, AF 532, AF 546, AF 555, AF 568, AF 594, AF 610, AF 633, AF 635, AF 647, AF 660, AF 680, AF 700, AF 750, AF 790), Allophycocyanin (APC), phycoerythrin (PE), Brilliant violet (BV) dye (e.g., BV 421, BV 480, BV 510, BV 570, BV 605, BV 650, BV 711, BV 750, BV 785), Peridinin-chlorophyll-protein (PerCP), Pacific blue, Pacific orange, PE/Dazzle 594, APC Fire 750, Super Bright 436, Zombie Green, Texas Red, Rhodamine, and cyanine (Cy) dye (e.g., Cy2, Cy3, Cy5).

Embodiment 20. The method of embodiment 10, wherein (c) detecting occurs by immunoassay.

Embodiment 21. The method of embodiment 20, wherein the immunoassay is selected from the group consisting of: lateral flow immunochromatography and/or enzyme-linked immunosorbent assay (ELISA).

Embodiment 22. The method of embodiment 20, wherein the immunoassay is selected from the group consisting of: enzyme immunoassay (EIA), ELISA, fluoroimmunoassay (FIA), chemiluminescent immunoassay (CLIA), counting immunoassay (CIA), and radioimmunoassay (RIA).

Embodiment 23. The method of embodiment 10, further comprising:
(i) measuring temperature of the subject (wherein the temperature is greater than (or equal to) 38° C.);
(ii) identifying an epidemiological link to SARS-COV-2 infection; and
(iii) identifying at least two of:
   (a) rash;
   (b) gastrointestinal symptoms;
   (c) edema of hands and/or feet;
   (d) oral mucosal changes;
   (e) conjunctivitis;
   (f) lymphoadenopathy; and
   (g) neurologic symptoms.

Embodiment 24. The method of embodiment 23, further comprising evaluating the

subject for other causes.

Embodiment 25. The method of embodiment 24, further comprising investigating for MIS-C.

Embodiment 26. The method of embodiment 25, further comprising identifying shock of an unclear etiology in the subject.

Embodiment 27. The method of embodiment 26, further comprising:
(iv) measuring C-reactive protein (CRP);
(v) measuring erythrocyte sedimentation rate (ESR);
(vi) measuring complete blood count (CBC);
(vii) measuring comprehensive metabolic panel (CMP);
(viii) testing for SARS-COV-2 infection (e.g., PCR; antigen; serology); and
(ix) measuring the at least one biomarker (e.g., FCGBP, MRC1, LBP, CD163; IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2), then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker is selected from any one of Group (B) of TABLE 4. either alone or in combination with any one of Group (A) of TABLE 3; and/or any combinations thereof).

Embodiment 28. The method of embodiment 27, wherein the at least one biomarker is selected from the group consisting of: FCGBP; MRC1; LBP; CD163; IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or at least one biomarker is selected from the group consisting of: Group (A) of TABLE 3 and/or Group (B) of TABLE 4, where if any one of the biomarkers of Group (A) of TABLE 3 is selected, then at least one biomarker of Group (B) of TABLE 4 is combined; or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); and any combinations thereof.

Embodiment 29. The method of embodiment 27, wherein:
(a) the C-reactive protein (CRP) level is 3 mg/dl or greater;
(b) the erythrocyte sedimentation rate (ESR) is 40 mm/hr or greater; 20 mm/hr to 50 mm/hr (normal from men 0 to 22 mm/hr (≤15 mm/hr); normal women 0 to 29 mm/hr (≤20 mm/hr); normal child ≤10 mm/hr);
(c) identifying at least one of:
   (1) absolute lymphocyte count of less than 1,000 μl;
   (2) platelet count of less than 150,000/μl;
   (3) sodium of less than 135 mmol/L;
   (4) neutrophilia; and
   (5) hypoalbuminemia; and
(d) identifying at least one of:
   (1) a mucosal immunity biomarker (e.g., mucins, Fc gamma binding protein (FCGBP), immunoglobulins (e.g., IgA, IgM), antimicrobial proteins (e.g., defensins, lysozyme), transferrins (e.g., lactoferrin));
   (2) a macrophage activation biomarker (e.g., Mannose receptor C-type 1 (MRC1), CD163, CD14, Gal-3BP, matrix metalloproteinases (e.g., MMP7, MMP9), cytokines); and
   (3) a bacterial infectivity indicator (e.g., white blood cell count, erythrocyte sedimentation rate, procalcitonin, C-reactive protein (CRP), serum amyloid A (SAA), cytokines, lipopolysaccharide (LPS) binding protein (LBP));
   (4) an intestinal damage/gut leakage biomarker (e.g., fatty acid binding protein (FABP), glucagon-like peptide (GLP)-2, citrulline, lipopolysaccharide (LPS), LPS-binding protein (LBP)); or
   (5) a biomarker selected from the group consisting of: IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or at least one biomarker is selected from the group consisting of: Group (A) of TABLE 3 and/or Group (B) of TABLE 4, where if any one of the biomarkers of Group (A) of TABLE 3 is selected, then at least one biomarker of Group (B) of TABLE 4 is combined; or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker is selected from any one of Group (B) of TABLE 4, either alone or in combination with any one of Group (A) of TABLE 3; and any combinations thereof.

Embodiment 30. The method of embodiment 29, further comprising at least one of:
(x) measuring B-type natriuretic peptide (BNP);
(xi) measuring troponin;
(xii) measuring procalcitonin;
(xiii) measuring ferritin;
(xiv) measuring prothrombin time (PT);
(xv) measuring partial thromboplastin time (PTT);
(xvi) measuring D-dimer;
(xvii) measuring fibrinogen;
(xviii) measuring lactate dehydrogenase (LDH);
(xix) measuring urinalysis (u/a);
(xx) measuring cytokine panel;
(xxi) measuring triglycerides;
(xxii) measuring electrocardiogram (EKG);
(xxiii) measuring echocardiogram; and
(xxiv) testing blood smear.

Embodiment 31. The method of embodiment 10, wherein the presence of at least one biomarker of (c) comprises a protein level of at least 2-fold change as compared to a protein level of a subject who does not suffer from MIS-C or severe acute COVID-19), or other negative control indicative of an absence of the at least one biomarker.

Embodiment 32. The method of embodiment 31, wherein the at least one biomarker is selected from the group consisting of: FCGBP; MRC1; LBP; CD163; IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or at least one biomarker is selected from the group consisting of: Group (A) of TABLE 3 and/or Group (B) of TABLE 4, where if any one of the biomarkers of Group (A) of TABLE 3 is selected, then at least one biomarker of Group (B) of TABLE 4 is combined; or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA 1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity (e.g., mucosal immunity (e.g., mucins, immunoglobulins (e.g., IgM), antimicrobial proteins (e.g., defensins, lysozyme), transferrins (e.g., lactoferrin)); macrophage activation (e.g., Mannose receptor C-type 1 (MRC1), Gal-3BP, matrix metalloproteinases (e.g., MMP7, MMP9)); bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker is selected from any one of Group (B) of TABLE 4, either alone or in combination with any one of Group (A) of TABLE 3; and any combinations thereof; and any combinations thereof.

Embodiment 33. The method of embodiment 10, wherein (d) treating comprises:
(i) administering at least one of: immunoglobulins (e.g., intravenous 2 g/kg (maximum of 100 g); IgG primarily without or with lower amounts of: IgA, IgD, IgE, IgM; PRIVIGEN®: 99.2% IgG. 0.8% monomers and dimers with IgA (1.8 mg/ml); GAMUNEX®-C: IgG/10% Caprylate/Chromatography Purified; intravenous: 300-600 mg/kg (1 mg/kg/min-8 mg/kg/min); subcutaneous: 1.37×current IV dose in g/IV dose interval in weeks; 10 mL/hr/site-15 mL/hr/site; 10 mL/hr/site-20 mL/hr/site per week; OCTAGAM® 5% liquid: 96% IgG, 50 mg IgG/ml; percents of total IgG: IgG1 is 65%, IgG2 is 30%, IgG3 is 3% and IgG4 is 2%)); steroids (e.g., intravenous 2-30 mg/kg/day methylprednisolone); immunomodulators or biologics (e.g., (e.g., anakinra 2-10 mg/kg/day via subcutaneous or intravenous, divided every 6 hrs to 12 hrs)); antibiotics; cytokine inhibitors; and any combination thereof in an effective amount sufficient to: reduce, alleviate, or slow the progression of a symptom of MIS-C, wherein the symptom of MIS-C is selected from the group consisting of: fever that lasts 24 hours or longer; vomiting; diarrhea; stomach pain; skin rash; fatigue; inability to wake up or stay awake; fast heartbeat; rapid breathing; difficulty breathing; red eyes; redness or swelling of the lips and tongue or hands or feet; pale lips or nail beds; headache, dizziness or lightheadedness; confusion; enlarged lymph nodes; and any combinations thereof.

Embodiment 34. The method of embodiment 33, wherein administering comprises: oral administration, intravenous administration, intramuscular administration, or combinations thereof.

Embodiment 35. The method of embodiment 33 or 34, wherein administering the combination occurs: together, separately, simultaneously (essentially), sequentially (essentially), or combinations thereof.

Embodiment 36. The method of embodiment 10, wherein the subject is a human.

Embodiment 37. The method of embodiment 10, wherein the apparatus (wherein the apparatus is configured for capture antibody coating, deposition, or conjugation) is selected from the group consisting of: a membrane (e.g., nitrocellulose; polyvinylidene fluoride (PVDF)); a multi-well plate (e.g., polystyrene, polyvinyl chloride); a bead; a chip (e.g., a microfluidic chip or a microchip); and a slide (e.g., glass) (wherein the apparatus is configured for capture antibody coating, deposition, or conjugation).

As various changes can be made in the above-described subject matter without departing from the scope and spirit of the present disclosure, it is intended that all subject matter contained in the above description, or defined in the appended embodiments and/or claims, be interpreted as descriptive and illustrative of the present disclosure. Many modifications and variations of the present disclosure are possible in light of the above teachings. Accordingly, the present disclosure is intended to embrace all such alternatives, modifications and variances which fall within the scope of the appended embodiments and/or claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

What is claimed is:

1. An apparatus, comprising:
at least one capture antibody attached thereto,
wherein the at least one capture antibody is configured to detect at least one biomarker for diagnosing and/or prognosing Multisystem Inflammatory Syndrome in Children (MIS-C) or severe acute COVID-19,
wherein the at least one biomarker is selected from the group consisting of:
at least one biomarker comprising a function selected from the group consisting of: mucosal immunity; macrophage activation; bacterial infectivity; intestinal damage/gut leakage; IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or
where if the biomarker is at least one selected from the group (A) of TABLE 3 consisting of: IgG Fc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2),
then the at least one biomarker of TABLE 3 is combined with at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; or
where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; or
wherein the at least one biomarker is selected from any one of Group (B) of TABLE 4, either alone or in combination with any one of Group (A) of TABLE 3; and any combinations thereof.

2. The apparatus of claim 1, wherein the at least one biomarker is selected from the group consisting of: mucosal immunity; Mannose receptor C-type 1 (MRC1), CD163, CD14, Gal-3BP, matrix metalloproteinases, cytokines; white blood cell count, erythrocyte sedimentation rate, procalcitonin, C-reactive protein (CRP), serum amyloid A (SAA), cytokines, lipopolysaccharide (LPS) binding protein (LBP); and any combinations thereof.

3. The apparatus of claim 2, wherein the at least one capture antibody is an antibody against the at least one biomarker.

4. The apparatus of claim 1, wherein the at least one capture antibody is against at least one biomarker of: Macrophage mannose receptor 1/Mannose receptor C-type 1 (MRC1) and/or Tryptophan-tRNA ligase, cytoplasmic (WARS1), either alone or in combination with an antibody against at least one biomarker selected from the group consisting of: a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; or
wherein the at least one capture antibody is against the at least one biomarker selected from any one of Group (B) of TABLE 4, either alone or in combination with any one of Group (A) of TABLE 3; and any combinations thereof.

5. The apparatus of claim 1, wherein the at least one capture antibody is selected from the group consisting of:
a monoclonal antibody against FCGBP; a monoclonal antibody against MRC1; a monoclonal antibody against LBP; a monoclonal antibody against CD163; a monoclonal antibody against IgGFc-binding protein/Fc gamma binding protein; a monoclonal antibody against Apolipoprotein C-III; a monoclonal antibody against Nidogen-1; a monoclonal antibody against Macrophage mannose receptor 1/Mannose receptor C-type 1; a monoclonal antibody against Cathepsin D; an antibody against Vascular cell adhesion protein 1; a monoclonal antibody against Junction plakoglobin; a monoclonal antibody against Proteasome subunit alpha type-5; a monoclonal antibody against Methanethiol oxidase; a monoclonal antibody against Ferritin light chain; a monoclonal antibody against Endoplasmin; an antibody against Proteasome subunit alpha type-1; a monoclonal antibody against Sulfhydryl oxidase 1; a monoclonal antibody against Proteasome subunit beta type-1; a monoclonal antibody against Tryptophan-tRNA ligase, cytoplasmic; a monoclonal antibody against Plasma kallikrein; a monoclonal antibody against Desmocollin-1; a monoclonal antibody against Alpha-1-antichymotrypsin; a monoclonal antibody against Thrombospondin-4; a monoclonal antibody against Prenylcysteine oxidase; a monoclonal antibody against Insulin-like growth factor binding protein 3; a monoclonal antibody against Insulin-like growth factor binding protein complex, acid labile subunit; a monoclonal antibody against Inter-alpha-trypsin inhibitor heavy chain H2; or
a monoclonal antibody against any one of the biomarker proteins identified in any one of TABLES 1-4;
where the at least one monoclonal antibody is against a biomarker, where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); insulin-like growth factor binding protein complex, acid labile subunit (IGFALS); Plasma kallikrein (KLKB1); Inter-alpha-trypsin inhibitor heavy chain H2 (ITIH2),
then the monoclonal antibody of the at least one biomarker of TABLE 3 is combined with a monoclonal antibody against at least one biomarker selected from the group (B) of TABLE 4 consisting of: Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; and any combinations thereof.

6. The apparatus of claim 1, wherein the at least one capture antibody comprises at least one monoclonal antibody,
where the monoclonal antibody is against at least one biomarker of: Macrophage mannose receptor 1/Mannose receptor C-type 1 (MRC1) and/or Tryptophan-tRNA ligase, cytoplasmic (WARS1), either alone or in combination with a monoclonal antibody against at least one biomarker selected from the group consisting of:
a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; or
at least one monoclonal antibody against the at least one biomarker selected from any one of Group (B) of TABLE 4, either alone or in combination with any one of Group (A) of TABLE 3; and any combinations thereof.

7. A method, comprising:
(a) contacting at least one capture antibody and a sample from a subject suspected of or suffering from Multisystem Inflammatory Syndrome in Children (MIS-C) or severe acute COVID-19, thereby forming a contacted sample on an apparatus;
(b) binding the contacted sample and a detection entity that binds to at least one biomarker of the sample;
(c) detecting the detection entity,
wherein the detection entity indicates the presence of at least one biomarker for Multisystem Inflammatory Syndrome in Children (MIS-C) or severe acute COVID-19 in the subject; and
(d) treating the subject for Multisystem Inflammatory Syndrome in Children (MIS-C) or severe acute COVID-19,
wherein the at least one biomarker comprises a function selected from the group consisting of: mucosal immunity; macrophage activation; intestinal damage/gut leakage; IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2;
where if the biomarker is at least one selected from the group (A) of TABLE 3 consisting of: IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); insulin-like growth factor binding protein complex, acid labile subunit (IGFALS); Plasma kallikrein (KLKB1); Inter-alpha-trypsin inhibitor heavy chain H2 (ITIH2),
then the at least one biomarker of TABLE 3 is combined with at least one biomarker selected from the group (B) of TABLE 4 consisting of:
Macrophage mannose receptor 1/Mannose receptor C-type 1 (MRC1); Tryptophan-tRNA ligase, cytoplasmic (WARS1); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity (e.g., white blood cell count, erythrocyte sedimentation rate, procalcitonin, serum amyloid A (SAA), intestinal damage/gut leakage (e.g., fatty acid binding protein (FABP), glucagon-like peptide (GLP)-2, citrulline, lipopolysaccharide (LPS)); or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or
a biomarker of Group (B) of TABLE 4 or
a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; or
the at least one biomarker is selected from any one of Group (B) of TABLE 4, either alone or in combination with any one of Group (A) of TABLE 3; and any combinations thereof.

8. The method of claim 7, wherein the apparatus comprises the at least one capture antibody, wherein the at least one capture antibody is configured to detect the at least one biomarker for diagnosing and/or prognosing Multisystem Inflammatory Syndrome in Children (MIS-C) or severe acute COVID-19.

9. The method of claim 7, wherein the sample comprises at least one biomarker for Multisystem Inflammatory Syndrome in Children (MIS-C) or severe acute COVID-19 in the subject.

10. The method of claim 7, further comprising reducing background interference on the apparatus.

11. The method of claim 7, wherein the detection entity comprises specificity for the at least one biomarker.

12. The method of claim 7, wherein the detection entity comprises specificity for the at least one capture antibody.

13. The method of claim 7, wherein the detection entity comprises a detectable label.

14. The method of claim 13, wherein the detectable label is selected from the group consisting of: an enzyme label, a fluorescent label, and a biotin label.

15. The method of claim 7, further comprising:
(i) measuring temperature of the subject;
(ii) identifying an epidemiological link to SARS-COV-2 infection; and
(iii) identifying at least two of:
(a) rash;
(b) gastrointestinal symptoms;
(c) edema of hands and/or feet;
(d) oral mucosal changes;
(e) conjunctivitis;
(f) lymphoadenopathy; and
(g) neurologic symptoms.

16. The method of claim 15, further comprising:
(iv) measuring C-reactive protein (CRP);
(v) measuring erythrocyte sedimentation rate (ESR);
(vi) measuring complete blood count (CBC);
(vii) measuring comprehensive metabolic panel (CMP);
(viii) testing for SARS-COV-2 infection; and
(ix) measuring the at least one biomarker.

17. The method of claim 16, wherein:
(a) the C-reactive protein (CRP) level is 3 mg/dl or greater;
(b) the erythrocyte sedimentation rate (ESR) is 40 mm/hr or greater; 20 mm/hr to 50 mm/hr (normal from men 0 to 22 mm/hr (≤15 mm/hr); normal women 0 to 29 mm/hr (≤20 mm/hr); normal child≤10 mm/hr);
(c) identifying at least one of:
(1) absolute lymphocyte count of less than 1,000 μl;
(2) platelet count of less than 150,000/μl;
(3) sodium of less than 135 mmol/L;
(4) neutrophilia; and
(5) hypoalbuminemia; and
(d) identifying at least one of:
(1) a mucosal immunity;
(2) a macrophage activation biomarker; and
(3) a bacterial infectivity indicator;
(4) an intestinal damage/gut leakage biomarker; or
(5) a biomarker selected from the group consisting of: IgGFc-binding protein/Fc gamma binding protein; Apolipoprotein C-III; Nidogen-1; Macrophage mannose receptor 1/Mannose receptor C-type 1; Cathepsin D; Vascular cell adhesion protein 1; Junction plakoglobin; Proteasome subunit alpha type-5; Methanethiol oxidase; Ferritin light chain; Endoplasmin; Proteasome subunit alpha type-1; Sulfhydryl oxidase 1; Proteasome subunit beta type-1; Tryptophan-tRNA ligase, cytoplasmic; Plasma kallikrein; Desmocollin-1; Alpha-1-antichymotrypsin; Thrombospondin-4; Prenylcysteine oxidase; Insulin-like growth factor binding protein 3; Insulin-like growth factor binding protein complex, acid labile subunit; Inter-alpha-trypsin inhibitor heavy chain H2; or at least one biomarker is selected from the group consisting of: Group (A) of TABLE 3 and/or Group (B) of TABLE 4, where if any one of the biomarkers of Group (A) of TABLE 3 is selected, then at least one biomarker of Group (B) of TABLE 4 is combined; or where the at least one biomarker comprises Macrophage mannose receptor 1/Mannose receptor C-type 1 (MRC1) and/or Tryptophan-tRNA ligase, cytoplasmic (WARS1), either alone or in combination with at least one biomarker (or at least one biomarker comprising a function) selected from the group consisting of a biomarker of: Group (A) of TABLE 3 and/or a biomarker of Group (B) of TABLE 4 and/or a biomarker of: LBP, CD163, zonulin, IgGFc-binding protein/Fc gamma binding protein (FCGBP); Alpha-1-antichymotrypsin (SERPINA3); Cathepsin D (CTSD); Ferritin light chain (FTL); Proteasome subunit alpha type-1 (PSMA1); Sulfhydryl oxidase 1 (QSOX1); Nidogen-1 (NID1); Apolipoprotein C-III (APOC3); insulin-like growth factor binding protein 3 (IGFBP3); and insulin-like growth factor binding protein complex, acid labile subunit (IGFALS); Plasma kallikrein (KLKB1); Inter-alpha-trypsin inhibitor heavy chain H2 (ITIH2); Vascular cell adhesion protein 1 (VCAM1); junction plakoglobin (JUP); Proteasome subunit alpha type-5 (PSMA5); Methanethiol oxidase (SELENBP1); Ferritin light chain (FTL); Endoplasmin (HSP90B1); Proteasome subunit beta type-1 (PSMB1); Desmocollin-1 (DSC1); Thrombospondin-4 (THBS4); Prenylcysteine oxidase (PCYOX1); mucosal immunity; macrophage activation; bacterial infectivity; or where the at least one biomarker is selected from any one of Group (B) of TABLE 4, either alone or in combination with any one of Group (A) of TABLE 3; and any combinations thereof.

18. The method of claim 17, further comprising at least one of:
(x) measuring B-type natriuretic peptide (BNP);
(xi) measuring troponin;
(xii) measuring procalcitonin;
(xiii) measuring ferritin;
(xiv) measuring prothrombin time (PT);
(xv) measuring partial thromboplastin time (PTT);
(xvi) measuring D-dimer;
(xvii) measuring fibrinogen;
(xviii) measuring lactate dehydrogenase (LDH);
(xix) measuring urinalysis (u/a);
(xx) measuring cytokine panel;
(xxi) measuring triglycerides;
(xxii) measuring electrocardiogram (EKG);
(xxiii) measuring echocardiogram; and
(xxiv) testing blood smear.

19. The method of claim 7, wherein the presence of at least one biomarker of (c) comprises a protein level of at least 2-fold change as compared to a protein level of a subject who does not suffer from MIS-C or severe acute COVID-19, or other negative control indicative of an absence of the at least one biomarker.

20. The method of claim 7, wherein (d) treating comprises:
administering at least one of: immunoglobulins; steroids; immunomodulators or biologics; antibiotics; cytokine inhibitors; and any combination thereof in an effective amount sufficient to: reduce, alleviate, or slow the progression of a symptom of MIS-C,
wherein the symptom of MIS-C is selected from the group consisting of: fever that lasts 24 hours or longer; vomiting; diarrhea; stomach pain; skin rash; fatigue; inability to wake up or stay awake; fast heartbeat; rapid breathing; difficulty breathing; red eyes; redness or swelling of the lips and tongue or hands or feet; pale lips or nail beds; headache, dizziness or lightheadedness; confusion; enlarged lymph nodes; and any combinations thereof.
